(19) United States
(12) Patent Application Publication
(10) Pub. No.: US 2009/0220515 A1
(43) Pub. Date: Sep. 3, 2009

(54) TREATMENT AND PREVENTION OF RENAL DISEASE

(75) Inventors: Michael J. Ross, Hastings-on-hudson, NY (US); Paul E. Klotman, New York, NY (US); Mary E. Klotman, New York, NY (US)

Correspondence Address: NIXON PEABODY LLP - PATENT GROUP, 1100 CLINTON SQUARE, ROCHESTER, NY, 14604, US

(73) Assignee: MOUNT SINAI SCHOOL OF MEDICINE, New York, NY (US)

(21) Appl. No.: 12/279,128

(22) PCT Filed: Feb. 13, 2007
(86) PCT No.: PCT/US07/62065

§ 371 Date: Dec. 18, 2008

Related U.S. Application Data
(60) Provisional application No. 60/772,753, filed on Feb. 13, 2006.

(51) Int. Cl. A61K 39/395; C12Q 1/68

(52) U.S. Cl. 424/139.1; 435/6

(57) ABSTRACT

The present invention is directed to a method of treating or preventing renal disease in a subject. This method involves administering the subject an agent which inhibits FAT10 expression in the subject under conditions effective to prevent or treat renal disease. Also disclosed is a method of screening for agents effective in treating or preventing renal disease.

This application claims the priority benefit of U.S. Provisional Patent Application Ser. No. 60/772,753, filed Feb. 13, 2006, which is hereby incorporated by reference in its entirety.

The present invention was made with funding under NIH Grant Nos. K08 DK062672 and P01 DK056492. The U.S. Government may have certain rights.

### FIELD OF THE INVENTION

The present invention relates to the treatment or prevention of renal disease.

### BACKGROUND OF THE INVENTION

Dysregulated epithelial apoptosis and proliferation contribute to the pathogenesis of several progressive renal diseases, including HIV-associated nephropathy (“HIVAN”) and autosomal dominant polycystic kidney disease (“ADPKD”) (Ross et al., “HIV-Associated Nephropathy,” Aids 18:1089-1099 (2004); Wilson, “Polycystic Kidney Disease,” N. Engl. J. Med. 350:151-164 (2004)). In both of these diseases, patients develop tubulointerstitial disease, characterized by the presence of tubular cysts in multiple nephron segments and interstitial fibrosis. In ADPKD, tubular cysts progressively enlarge, eventually leading to compression of the remaining normal renal parenchyma (Wilson, “Polycystic Kidney Disease,” N. Engl. J. Med. 350:151-164 (2004)). In HIVAN, cyst development ceases at the “microcystic” stage (D'Agati et al., “Pathology of HIV-Associated Nephropathy: A Detailed Morphologic and Comparative Study,” Kidney Int. 35:1358-1370 (1989)).

HIV-1 infection of the renal epithelium and subsequent epithelial expression of viral genes is a crucial determinant of HIVAN pathogenesis (Bruggeman et al., “Nephropathy in Human Immunodeficiency Virus-1 Transgenic Mice is Due to Renal Transgene Expression,” J. Clin. Invest. 100:84-92 (1997); Bruggeman et al., “Renal Epithelium is a Previously Unrecognized Site of HIV-1 Infection,” J. Am. Soc. Nephrol. 11:2079-2087 (2000)). Epithelial apoptosis induced by expression of HIV-1 genes is an important contributor to progression of renal failure (Bruggeman et al., “Nephropathy in Human Immunodeficiency Virus-1 Transgenic Mice is Due to Renal Transgene Expression,” J. Clin. Invest. 100:84-92 (1997); Conaldi et al., “HIV-1 Kills Renal Tubular Epithelial Cells In Vitro by Triggering an Apoptotic Pathway Involving Caspase Activation and Fas Upregulation,” J. Clin. Invest. 102:2041-2049 (1998); Bodi et al., “Apoptosis in Human Immunodeficiency Virus-Associated Nephropathy,” Am. J. Kidney Dis. 26:286-291 (1995)). In patients with HIVAN, HIV-1 infection occurs in epithelial cells from any segment of the nephron, including the glomerulus, proximal tubule, thick ascending limb of Henle, and collecting duct (Ross et al., “Microcyst Formation and HIV-1 Gene Expression Occur in Multiple Nephron Segments in HIV-Associated Nephropathy,” J. Am. Soc. Nephrol. 12:2645-2651 (2001)). While HIV-1 infection of renal tubular epithelium is known to occur, the host genes that are differentially expressed following infection by HIV-1 that subsequently lead to proliferation and apoptosis remain largely undefined.

There clearly are genetic factors that contribute to HIVAN susceptibility. HIVAN occurs almost exclusively in individuals of African ancestry and blacks with HIVAN are far more likely to have HIV-negative relatives with end stage renal disease (“ESRD”) than are black persons without HIVAN (Freedman et al., “Familial Clustering of End-Stage Renal Disease in Blacks with HIV-Associated Nephropathy,” Am. J. Kidney Dis. 34:254-258 (1999)). Moreover, in the HIV-1 transgenic model of HIVAN, the penetrance of the HIVAN phenotype is dramatically influenced by the genetic background of the mice (Gharavi et al., “Mapping a Locus for Susceptibility to HIV-1-Associated Nephropathy to Mouse Chromosome 3,” Proc. Natl. Acad. Sci. USA 101:2488-2493 (2004); Gharavi et al., “Genetic Background Modifies the Development of Renal Disease in HIV-1 Transgenic Mice,” J. Amer. Soc. Nephrol. 10:404A (1999)). Despite the importance of host factors, most in vitro studies of HIVAN pathogenesis have used cell lines that were derived either from animals or humans from racial groups with a very low susceptibility to developing HIVAN.

The present invention is directed to overcoming these and other limitations in the art.

### SUMMARY OF THE INVENTION

One aspect of the present invention is directed to a method of treating or preventing renal disease in a subject. This method involves administering to the subject an agent which inhibits FAT10 expression in the subject under conditions effective to prevent or treat renal disease.

Another aspect of the present invention is directed to a method of screening for agents effective in treating or preventing renal disease. This method involves providing one or more candidate compounds. The one or more candidate compounds are contacted with a FAT10 gene under conditions effective to express the FAT10 gene in the absence of the one or more candidate compounds. Candidate compounds that inhibit FAT10 expression are identified as agents potentially useful in treating or preventing renal disease.

In studies using a proximal tubular cell line derived from a patient with HIVAN, oligonucleotide microarrays were used to determine the genes that are differentially expressed following transduction with replication-incompetent HIV-1. In these studies, which are described herein, one of the most robustly upregulated genes was FAT10. FAT10 is a ubiquitin-like protein, containing two ubiquitin-like domains, that is capable of inducing ubiquitin-independent degradation of proteins via the proteasome (Hipp et al., “FAT10, A Ubiquitin-Independent Signal for Proteasomal Degradation,” Mol. Cell. Biol. 25:3483-3491 (2005), which is hereby incorporated by reference in its entirety). Since FAT10 has putative roles in regulating apoptosis and cell cycle progression (Liu et al., “A MHC-Encoded Ubiquitin-Like Protein (FAT10) Binds Noncovalently to the Spindle Assembly Checkpoint Protein MAD2,” Proc. Natl. Acad. Sci. USA 96:4313-4318 (1999); Raasi et al., “The Ubiquitin-Like Protein FAT10 Forms Covalent Conjugates and Induces Apoptosis,” J. Biol. Chem. 276:35334-35343 (2001), which are hereby incorporated by reference in their entirety), FAT10 expression was studied in the HIV-1 transgenic model of HIVAN and in HIVAN biopsy specimens. Given that ADPKD is also characterized by renal tubular dilatation and dysregulated apoptosis Nadasdy et al., “Proliferative Activity of Cyst Epithelium in Human Renal Cystic Diseases,” J. Am. Soc. Nephrol. 5:1462-1468 (1995); Woo, “Apoptosis and Loss of Renal Tissue in Polycystic Kidney Diseases,” N. Engl. J. Med. 333:18-25 (1995), which are hereby incorporated by reference in their entirety), the expression of FAT10 was examined in ADPKD. As described infra, FAT10 expression is increased in renal epithelial cells in HIVAN and ADPKD, in vitro expression of FAT10 induces apoptosis in human renal tubular epithelial cells (“RTECs”), and inhibition of FAT10 expression prevents HIV-induced apoptosis in RTECs.

### BRIEF DESCRIPTION OF THE DRAWINGS

FIG. 1 is a schematic illustration of the FAT10 protein, containing two ubiquitin homology domains and a C-terminal diglycine motif.

FIGS. 2A-F are schematic representations of constructs used in the present invention. FIG. 2A is a schematic illustration of the construct pHR′-tsTAg-IRES-hygro, which was used to produce lentivirus to conditionally immortalize proximal tubular epithelial cells (“PTECs”). FIG. 2B is a schematic illustration of the construct pNL4-3:ΔG/P-EGFP, which encodes a gag/pol-deleted HIV-1 provirus with enhanced green fluorescent protein (“EGFP”) cloned into the gag open reading frame. pNL4-3:ΔG/P-EGFP and the construct pHR′-IRES-EGFP (schematically illustrated in FIG. 2C) were used to generate VSV-G-pseudotyped lentiviruses for infection of HPT-1 cells. FIG. 2D is a schematic illustration of the construct pVVPW/BE-FAT10, which is a bicistronic vector expressing FAT10 and EGFP as separate proteins. FIG. 2E is a schematic illustration of the construct pcDNA4-FAT10, which expresses FAT10 with N-terminal histidine and Xpress epitope tags. FIG. 2F is a schematic illustration of the construct pVIRHD/E, which is a lentiviral vector that allows production of shRNA in transduced cells.

FIGS. 3A-D are images illustrating that HPT-1 cells express temperature-sensitive T-antigen when grown at 33° C.; but at 37° C., T-antigen degrades (FIG. 3A) and the cells differentiate, expressing the epithelial marker cytokeratin (FIG. 3B, magnification ×400) and the proximal tubular markers alkaline phosphatase (FIG. 3C, magnification ×200) and aquaporin-1 (FIG. 3D, magnification ×200).

FIGS. 4A-B are images showing that FAT10 mRNA expression is increased in HIV-infected HPT-1 cells and in kidneys from HIV-transgenic mice. Infection with gag/pol-deleted HIV-1 induced expression of FAT10 at 3 and 7 days after infection (FIG. 4A). Expression of FAT10 mRNA was markedly increased in severely diseased kidneys from HIV-transgenic mice (FIG. 4B). Lesser expression of FAT10 was present in kidneys from transgenic mice without overt nephropathy and there was minimal expression in kidneys from normal adult mice (FIG. 4B).

FIGS. 5A-C are graphs showing that FAT10 expression induces apoptosis in HPT-1 cells. 24 hours after transfection with VVPW/BE-FAT10, EGFP-expressing HPT-1 cells were examined by flow cytometry. 18.1% of cells transfected with pVVPW/BE-FAT10 were apoptotic as determined by annexin V staining (FIGS. 5A and 5C), whereas only 1.8% of vector-transfected cells became apoptotic (FIGS. 5B and 5C; p<0.0001). 47.3% of HPT-1 cells transfected with FAT10 were either apoptotic (annexin V positive, propidium negative) or necrotic (positive for propidium) in contrast to vector-transfected cells (9.3%, FIG. 5C) (p<0.0001).

FIGS. 6A-B show that stable transduction of HPT-1 cells with FAT10 shRNA constructs (VIRHD/E-FAT10.1 and VIRHD/E-FAT10.2) markedly reduces FAT10 protein production after transfection with a FAT10 expression vector as compared to HPT-1 cells not expressing FAT10 shRNA (FIG. 6A). HPT-1 cells in which endogenous FAT10 expression is suppressed by stable transduction with FAT10 shRNA (VIRHD/E-FAT10.1 and VIRHD/E-FAT10.2) are resistant to apoptosis after infection with VSV-NL4-3-ΔG/P-EGFP as compared to HPT-1 cells expressing the luciferase shRNA vector VIRHD/E/siLuc (FIG. 6B, * p-value<0.0001).

FIGS. 7A-H are photographs showing FAT10 protein expression in kidneys from HIV-1 transgenic mice with HIVAN. FAT10 is detected in tubular epithelial cells (FIG. 7A), in tubular epithelial cells that have detached from the tubular basement membrane (FIG. 7B) and in cells beneath the tubular epithelium (FIG. 7C, arrow). Focal expression of FAT10 was also detected in glomeruli (FIG. 7D) and in vascular smooth muscle cells (FIG. 7E). No FAT10 protein was detected in normal adult murine kidneys (FIG. 7F) with the exception of some vascular smooth muscle cells. Preimmune serum controls using kidneys from transgenic mice are negative (FIG. 7G) and preincubation of antiserum with 10 ng/mL of immunizing peptide abolished FAT10 staining in transgenic kidneys (FIG. 7H). Magnification ×200 (FIGS. 7A, 7C, 7E, 7F, 7G, and 7H) and ×400 (FIGS. 7B and 7D).

FIGS. 8A-K are photographs showing FAT10 expression in HIVAN kidney biopsies and in ADPKD. As shown in FIGS. 8A-F, FAT10 is expressed in fibrotic scars surrounding sclerotic glomeruli (FIG. 8A), in tubular epithelial cells (FIG. 8B), and in vascular smooth muscle cells (FIG. 8C). Preimmune controls performed on serial sections (FIGS. 8D-F) were negative. Magnification ×400 (FIGS. 8A, C, D, F) and ×200 (FIGS. 8B and E). As shown in FIGS. 8G-J, FAT10 expression is increased in cystic epithelium in ADPKD. FAT10 protein was detected in the epithelium lining cysts (FIGS. 8G and H) and in areas of aberrant epithelial proliferation (FIG. 8H, arrowheads). Preimmune serum controls demonstrated no staining (FIGS. 8I and J). As shown in FIG. 8K, FAT10 was not detected in normal human nephrectomy specimen. Magnification ×100 (FIGS. 8A, B, C, G, H), ×400 (FIGS. 8D, E, F, I, J), and ×200 (FIG. 8K).

FIGS. 9A-F is a series of photographs from immunohistochemistry experiments conducted to determine if FAT10 is expressed in renal biopsy specimens from patients with diabetic nephropathy (FIGS. 9A-B), hypertensive nephrosclerosis (FIGS. 9C-D), and IgA nephropathy (FIGS. 9E-F). A striking expression of FAT10 was observed in 15/15 specimens from patients with each of these diseases (5 patients with each disease). FAT10 was detected predominantly in RTEC, with some glomerular expression, especially in diabetic nephropathy (FIGS. 9A-B).

FIGS. 10A-B illustrate nonsynonymous single nucleotide polymorphisms (“SNPs”) and haplotypes in FAT10. FIG. 10A is a schematic illustration of FAT10 and the location of six nonsynonymous SNPs. FIG. 10B is a table identifying four FAT10 haplotypes.

FIG. 11 is a graph showing that the FAT10^{b} allele is more prevalent in patients with HIVAN than in HIV-positive patients with non-HIVAN renal disease, even after controlling for ethnicity and sex (p=0.01).

FIG. 12 is a graph showing that expression of FAT10^{b} induces greater levels of apoptosis in human RTEC than other alleles of FAT10 (*p<0.0001 for all alleles vs. FAT10^{b}).

FIGS. 13A-B illustrate that FAT10 alleles differ in their ability to form high molecular weight conjugates with FAT10^{c} forming the most and FAT10^{b} forming the least conjugate.

### DETAILED DESCRIPTION OF THE INVENTION

One aspect of the present invention is directed to a method of treating or preventing renal disease in a subject. This method involves administering to the subject an agent which inhibits FAT10 expression in the subject under conditions effective to prevent or treat renal disease.

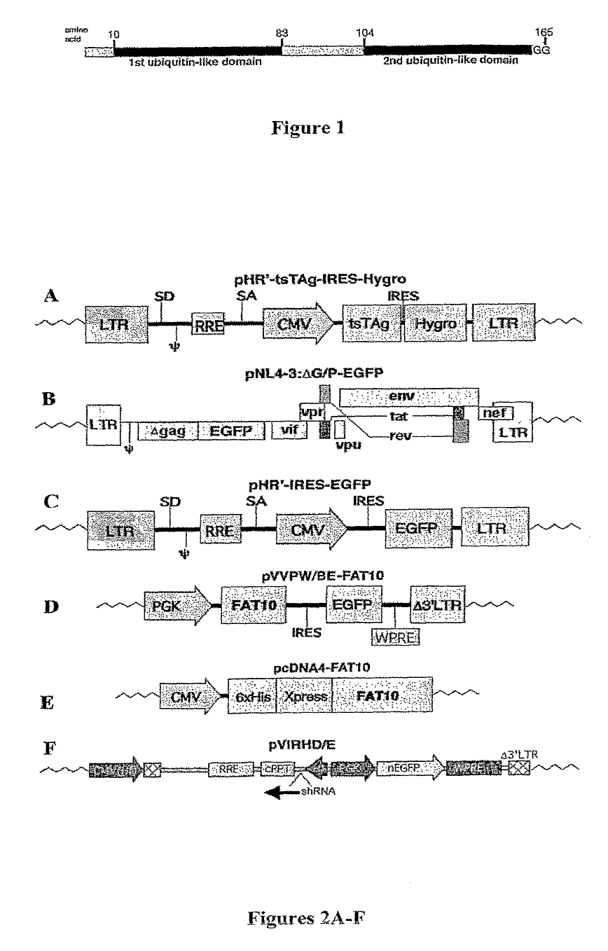

FAT10 is a 165 amino acid protein with two ubiquitin-like domains and a conserved diglycine motif at its C-terminus (FIG. 1). This C-terminal diglycine is necessary for the covalent conjugation of ubiquitin and ubiquitin like proteins (“UBLs”) to target proteins, suggesting that FAT10 may become covalently conjugated to other proteins.

As reported at Genbank Accession No. AF123050, which is hereby incorporated by reference in its entirety, FAT10 has an amino acid sequence corresponding to SEQ ID NO:1 as follows:

MAPNASCLCV HVRSEEWDLM TFDANPYDSV KKIKEHVRSK TKVPVQDQVL LLGSKILKPR
RSLSSYGIDK EKTIHLTLKV VKPSDEELPL FLVESGDEAK KHLLQVRRSS SVAQVKAMIE
TKTGIIPETQ IVTCNGKRLE DGKMMADYGI RKGNLLFLAS YCIGG

The FAT10 gene has a nucleotide sequence corresponding to SEQ ID NO:2 as follows:

| | |
|---|---|
| 1 | gattgcttga ggagagaagt atgtgatcag aaagcattct ttgtctatta actcctgccc |
| 61 | agcaaaagtg aaagaaaatt catgggagca tgcaagaaca aagagcacag caaagctgga |
| 121 | caaacacagc aatccaggca ggggatttcc aactcaactc tggtatgtaa gctgcatgca |
| 181 | aagtcctttt tctgtctctg gtttctggcc ccttgtctgc agagatggct cccaatgctt |
| 241 | cctgcctctg tgtgcatgtc cgttccgagg aatgggattt aatgaccttt gatgccaacc |
| 301 | catatgacag cgtgaaaaaa atcaaagaac atgtccggtc taagaccaag gttcctgtgc |
| 361 | aggaccaggt tcttttgctg ggctccaaga tcttaaagcc acggagaagc ctctcatctt |
| 421 | acggcattga caaagagaag accatccacc ttaccctgaa agtggtgaag cccagtgatg |
| 481 | aggagctgcc cttgtttctt gtggagtcag gtgatgaggc aaagaggcac ctcctccagg |
| 541 | tgcgaaggtc cagctcagtg gcacaagtga aagcaatgat cgagactaag acgggtataa |
| 601 | tccctgagac ccagattgtg acttgcaatg gaaagagact ggaagatggg aagatgatgg |
| 661 | cagattacgg catcagaaag ggcaacttac tcttcctggc atcttattgt attggagggt |
| 721 | gaccaccctg ggcatggggt gttggcaggg gtcaaaaagc ttatttcttt taatctctta |
| 781 | ctcaacgaac acatcttctg atgatttccc aaaattaatg agaatgagat gagtagagta |
| 841 | agatttgggt gggatgggta ggatgaagta tattgcccaa ctctatgttt ctttgattct |
| 901 | aacacaatta attaagtgac atgattttta ctaatgtatt actgagacta gtaaataaat |
| 961 | ttttaagcca a |

Renal diseases associated with undesirable FAT10 expression or activity include both chronic and acute renal diseases or disorders. Particular renal diseases or disorders amenable to the method of the present invention are autosomal dominant polycystic kidney disease, HIV-associated nephropathy, diabetic nephropathy, hypertensive nephrosclerosis, IgA nephropathy, and acute rejection of transplanted kidneys. In a particular embodiment, the renal disease is characterized by dysregulated apoptosis of renal tubular epithelial cells.

The subject to whom the agent is to be administered can be any mammal, preferably human.

Agents (or compositions) which inhibit FAT10 expression can be administered in any manner that is effective to deliver the agent anywhere FAT10 expression or activity is intended to be modified (e.g., affected renal epithelial cells). This can be accomplished either via systemic administration to the subject or via targeted administration to affected cells. Exemplary routes of administration include, without limitation, orally, by inhalation, by intranasal instillation, topically, transdermally, parenterally, subcutaneously, intravenous injection, intra-arterial injection (such as via the pulmonary artery), intramuscular injection, intrapleural instillation, intraperitoneally injection, intraventricularly, intralesionally, by application to mucous membranes, or implantation of a sustained release vehicle adjacent to affected cells.

The administration of the therapeutic agent can be carried out as frequently as required and for a duration that is suitable to provide effective treatment for the disease conditions being treated. For example, administration of the therapeutic agent can be carried out with a single sustained-release dosage formulation or with multiple daily doses of the therapeutic agent. The amount to be administered will, of course, vary depending upon the treatment regimen.

Typically, the therapeutic agent will be administered to a subject as a pharmaceutical composition that includes the therapeutic agent and any pharmaceutically acceptable suitable adjuvants, carriers, excipients, or stabilizers, and can be in solid or liquid form such as, tablets, capsules, powders, solutions, suspensions, or emulsions. The compositions preferably contain from about 0.01 to 99 weight percent, more preferably from about 2 to 60 percent, of therapeutic agent together with the adjuvants, carriers and/or excipients. The amount of active compound in such therapeutically useful compositions is such that a suitable dosage unit will be obtained. Preferred compositions according to the present invention are prepared so that a single dosage unit contains between about 1 mg and 1000 mg of the therapeutic agent.

In one aspect, the invention provides a method for preventing in a subject, a disease or condition associated with FAT10 expression or activity, by administering to the subject an effective amount of an agent that modulates (i.e., inhibits) FAT10 expression. Subjects at risk for a disease which is caused or contributed to by aberrant FAT10 expression or activity can be identified by, for example, any or a combination of diagnostic or prognostic assays that are known in the art. Prophylactic administration of an agent can occur prior to the manifestation of symptoms characteristic of the FAT10 aberrancy, such that a disease or disorder is prevented or, alternatively, delayed in its progression. The appropriate agent can be determined based on screening assays described herein.

Inhibiting FAT10 expression or activity involves contacting a cell with an agent that inhibits FAT10 expression or activity. An agent that inhibits FAT10 protein activity can be any agent as described herein, such as a nucleic acid or a protein (e.g., an anti-FAT10 antibody) as described infra, a naturally-occurring cognate ligand of FAT10, a peptide, a peptidomimetic, or other small molecule.

According to one approach, an agent that inhibits expression of FAT10 can be administered to a subject in a therapeutically effective amount and in a manner suitable to inhibit expression of FAT10 in renal epithelial cells, thereby treating the renal disease condition. One example of such an agent is siRNA targeted to the FAT10 nucleotide sequence, which interferes with translation of the FAT10 protein. siRNA for FAT10 can be selected using the online GenScript Corp. service, Promega Corp. service, or Ambion Inc. service.

The siRNA can be administered to the subject systemically as described herein or otherwise known in the art. Systemic administration can include those described above, but preferably intravenous, intraarterial, subcutaneous, intramuscular, catheterization, or nasopharangeal as is generally known in the art. Alternatively, the siRNA can be administered to the subject locally or to local tissues as described herein or otherwise known in the art. Local administration can include, for example, catheterization, implantation, direct injection, dermal/transdermal application, stenting, ear/eye drops, or portal vein administration to relevant tissues, or any other local administration technique, method or procedure, as is generally known in the art.

Delivery of siRNA is preferably administered alone or as a component of a composition. Suitable compositions include the siRNA formulated or complexed with polyethylenimine (e.g., linear or branched PEI) and/or polyethylenimine derivatives, including for example grafted PEIs such as galactose PEI, cholesterol PEI, antibody derivatized PEI, and polyethylene glycol PET (PEG-PET) derivatives thereof (see, e.g., Ogris et al., AAPA Pharm. Sci. 3:1-11 (2001), Furgeson et al., Bioconjugate Chem. 14:840-847 (2003); Kunath et al., Pharmaceutical Res. 19: 810-817 (2002); Choi et al., Bull. Korean Chem. Soc. 22:46-52 (2001); Bettinger et al., Bioconjugate Chem. 10:558-561 (1999); Peterson et al., Bioconjugate Chem. 13:845-854 (2002); Erbacher et al., J. Gene Medicine Preprint 1:1-18 (1999); Godbey et al., Proc. Natl. Acad. Sci. USA 96:5177-5181 (1999); Godbey et al., J. Controlled Release 60:149-160 (1999); Diebold et al., J. Biol. Chem. 274:19087-19094 (1999); Thomas et al., Proc. Natl. Acad. Sci. USA 99:14640-14645 (2002); and U.S. Pat. No. 6,586,524 to Sagara, which are hereby incorporated by reference in their entirety.

The siRNA molecule can also be present in the form of a bioconjugate, for example a nucleic acid conjugate as described in U.S. Pat. No. 6,528,631, U.S. Pat. No. 6,335,434, U.S. Pat. No. 6,235,886, U.S. Pat. No. 6,153,737, U.S. Pat. No. 5,214,136, or U.S. Pat. No. 5,138,045, which are hereby incorporated by reference in their entirety.

The siRNA, or any composition or bioconjugate containing the same, can be administered via a liposomal delivery mechanism. Basically, this involves providing a liposome which includes the siRNA to be delivered, and then contacting the target cell with the liposome under conditions effective for delivery of the siRNA into the cell.

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner where the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Proc. Natl. Acad. Sci. USA 84:7851 (1987); Biochemistry 28:908 (1989), which are hereby incorporated by reference in their entirety). When liposomes are endocytosed by a target cell, for example, they can be routed to acidic endosomes which will destabilize the liposome and result in drug release.

The liposome delivery system can also be made to accumulate at a target organ, tissue, or cell via active targeting (e.g., by incorporating an antibody or hormone on the surface of the liposomal vehicle). This can be achieved according to known methods.

Different types of liposomes can be prepared according to Bangham et al., J. Mol. Biol. 13:238-252 (1965); U.S. Pat. No. 5,653,996 to Hsu et al.; U.S. Pat. No. 5,643,599 to Lee et al.; U.S. Pat. No. 5,885,613 to Holland et al.; U.S. Pat. No. 5,631,237 to Dzau et al.; and U.S. Pat. No. 5,059,421 to Loughrey et al., which are hereby incorporated by reference in their entirety.

These liposomes can be produced such that they contain, in addition to siRNA, other therapeutic agents, such as anti-inflammatory agents, which would then be released at the target site (e.g., Wolff et al., Biochem. et Biophys. Acta 802:259 (1984), which is hereby incorporated by reference in its entirety).

As an alternative to non-infective delivery of the inhibitory RNA as described above, naked DNA or infective transformation vectors can be used for delivery, whereby the naked DNA or infective transformation vector contains a recombinant gene that encodes the inhibitory RNA capable of inhibiting expression of FAT10. The inhibitory RNA molecule is then expressed in the transformed cell.

The recombinant gene includes, operatively coupled to one another, an upstream promoter operable in mammalian cells and optionally other suitable regulatory elements (i.e., enhancer or inducer elements), a coding sequence that encodes the therapeutic nucleic acid, and a downstream transcription termination region. Any suitable constitutive promoter or inducible promoter can be used to regulate transcription of the recombinant gene, and one of skill in the art can readily select and utilize such promoters, whether now known or hereafter developed. The promoter can also be specific for expression in renal epithelial cells. Tissue specific promoters can also be made inducible/repressible using, e.g., a TetO response element. Other inducible elements can also be used. Known recombinant techniques can be utilized to prepare the recombinant gene, transfer it into the expression vector (if used), and administer the vector or naked DNA to a patient. Exemplary procedures are described below.

Any suitable viral or infective transformation vector can be used. Exemplary viral vectors include, without limitation, adenovirus, adeno-associated virus, and retroviral vectors (including lentiviral vectors).

Adenovirus gene delivery vehicles can be readily prepared and utilized given the disclosure provided in Berkner, Biotechniques 6:616-627 (1988), Rosenfeld et al., Science 252:431-434 (1991), WO 93/07283, WO 93/06223, and WO 93/07282, which are hereby incorporated by reference in their entirety. Additional types of adenovirus vectors are described in U.S. Pat. No. 6,057,155 to Wickham et al., U.S. Pat. No. 6,033,908 to Bout et al., U.S. Pat. No. 6,001,557 to Wilson et al., U.S. Pat. No. 5,994,132 to Chamberlain et al., U.S. Pat. No. 5,981,225 to Kochanek et al., U.S. Pat. No. 5,885,808 to Spooner et al., and U.S. Pat. No. 5,871,727 to Curiel, which are hereby incorporated by reference in their entirety.

Adeno-associated viral gene delivery vehicles can be constructed and used to deliver into cells a recombinant gene encoding a desired nucleic acid. The use of adeno-associated viral gene delivery vehicles in vitro is described in Chatterjee et al., Science 258:1485-1488 (1992); Walsh et al., Proc. Nat'l Acad. Sci. USA 89:7257-7261 (1992); Walsh et al., J. Clin. Invest. 94:1440-1448 (1994); Flotte et al., J. Biol. Chem. 268:3781-3790 (1993); Ponnazhagan et al., J. Exp. Med. 179:733-738 (1994); Miller et al., Proc. Nat'l Acad. Sci. USA 91:10183-10187 (1994); Einerhand et al., Gene Ther. 2:336-343 (1995); Luo et al., Exp. Hematol. 23:1261-1267 (1995); and Zhou et al., Gene Ther. 3:223-229 (1996), which are hereby incorporated by reference in their entirety. In vivo use of these vehicles is described in Flotte et al., Proc. Nat'l Acad. Sci. USA 90:10613-10617 (1993); and Kaplitt et al., Nature Genet. 8:148-153 (1994), which are hereby incorporated by reference in their entirety.

Retroviral vectors which have been modified to form infective transformation systems can also be used to deliver a recombinant gene encoding a desired nucleic acid product into a target cell. One such type of retroviral vector is disclosed in U.S. Pat. No. 5,849,586 to Kriegler et al., which is hereby incorporated by reference in its entirety. Lentivirus vectors can also be utilized, including those described in U.S. Pat. No. 6,790,657 to Arya, and U.S. Patent Application Nos. 20040170962 to Kafri et al. and 20040147026 to Arya, which are hereby incorporated by reference in their entirety.

The method of treating or preventing renal disease of the present invention may also involve an RNA-based form of gene-silencing known as RNA-interference (RNAi). Numerous reports have been published on critical advances in the understanding of the biochemistry and genetics of both gene silencing and RNAi (Matzke et al., “RNA-Based Silencing Strategies in Plants,” Curr. Opin. Genet. Dev. 11(2):221-227 (2001), which is hereby incorporated by reference in its entirety). In RNAi, the introduction of double stranded RNA (dsRNA, or iRNA, for interfering RNA) into animal or plant cells leads to the destruction of the endogenous, homologous mRNA, phenocopying a null mutant for that specific gene. In both post-transcriptional gene silencing and RNAi, the dsRNA is processed to short interfering molecules of 21-, 22- or 23-nucleotide RNAs (siRNA) by a putative RNAaseIII-like enzyme (Tuschl T., “RNA Interference and Small Interfering RNAs,” Chembiochem 2:239-245 (2001); Zamore et al., “RNAi: Double Stranded RNA Directs the ATP-Dependent Cleavage of mRNA at 21 to 23 Nucleotide Intervals,” Cell 101:25-3, (2000), each of which is hereby incorporated by reference in its entirety). The endogenously generated siRNAs mediate and direct the specific degradation of the target mRNA. In the case of RNAi, the cleavage site in the mRNA molecule targeted for degradation is located near the center of the region covered by the siRNA (Elbashir et al., “RNA Interference is Mediated by 21- and 22-Nucleotide RNAs,” Gene Dev. 15(2):188-200 (2001), which is hereby incorporated by reference in its entirety). The dsRNA for the nucleic acid molecule of the present invention can be generated by transcription in vivo, which involves modifying the nucleic acid molecule encoding FAT10 for the production of dsRNA, inserting the modified nucleic acid molecule into a suitable expression vector having the appropriate 5′ and 3′ regulatory nucleotide sequences operably linked for transcription and translation, and introducing the expression vector having the modified nucleic acid molecule into a suitable host cell or subject. Alternatively, complementary sense and antisense RNAs derived from a substantial portion of the coding region of the FAT10 nucleic acid molecule are synthesized in vitro (Fire et al., “Specific Interference by Ingested dsRNA,” Nature 391:806-811 (1998); Montgomery et al, “RNA as a Target of Double-Stranded RNA-Mediated Genetic Interference in Caenorhabditis elegans,” Proc. Natl. Acad. Sci. USA 95:15502-15507; Tabara et al., “RNAi in C. elegans: Soaking in the Genome Sequence,” Science 282:430-431 (1998), which are hereby incorporated by reference in their entirety). The resulting sense and antisense RNAs are annealed in an injection buffer, and dsRNA is administered to the subject using any method of administration described herein, supra.

In another embodiment, the agent is an antibody raised against a peptide encoded by the FAT10 gene. The antibody is capable of binding to at least a portion of a human FAT10 protein or peptide and functions to inhibit activity of such protein or peptide. Such antibodies may have therapeutic potential, particularly in the treatment of renal disease by inhibiting expression of FAT10 (e.g., by inhibiting function of a FAT10 protein or peptide). Particular peptides against which such an antibody may be raised are the peptides APNASCLCVHVRSE (SEQ ID NO:3) and DANPYDSVKKIKEVHR (SEQ ID NO:4), which are encoded by the FAT10 gene.

Antibodies may be either monoclonal antibodies or polyclonal antibodies. Monoclonal antibody production may be carried out by techniques which are well-known in the art. Basically, the process involves first obtaining immune cells (lymphocytes) from the spleen of a mammal (e.g., mouse) which has been previously immunized with the antigen of interest either in vivo or in vitro. The antibody-secreting lymphocytes are then fused with (mouse) myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler et al., Nature 256:495 (1975), which is hereby incorporated by reference in its entirety.

Mammalian lymphocytes are immunized by in vivo immunization of the animal (e.g., a mouse) with the protein or polypeptide of the present invention. Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by standard and well-known techniques, for example, by using polyethylene glycol or other fusing agents (Milstein et al., Eur. J. Immunol. 6:511 (1976), which is hereby incorporated by reference in its entirety). This immortal cell line, which is preferably murine, but may also be derived from cells of other mammalian species, including but not limited to rats and humans, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies are also well known. Typically, such antibodies can be raised by administering the protein or polypeptide of the present invention subcutaneously to New Zealand white rabbits which have first been bled to obtain pre-immune serum. The antigens can be injected at a total volume of 100 μl per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthenized with pentobarbital 150 mg/Kg IV. This and other procedures for raising polyclonal antibodies are disclosed in Harlow et. al., editors, Antibodies: A Laboratory Manual (1988), which is hereby incorporated by reference in its entirety.

In addition to utilizing whole antibodies, the present invention may employ the use of binding portions of such antibodies. Such binding portions include Fab fragments, F(ab′)₂ fragments, and Fv fragments. These antibody fragments can be made by conventional procedures, such as proteolytic fragmentation procedures, as described in Goding, Monoclonal Antibodies: Principles and Practice, pp. 98-118 (N.Y. Academic Press, 1983), which is hereby incorporated by reference in its entirety.

Many routes of delivery are known to the skilled artisan for delivery of anti-target antibodies. For example, direct injection may be suitable for delivering the antibody to the site of interest. It is also possible to utilize liposomes with antibodies in their membranes to specifically deliver the liposome to the area where FAT10 expression or function is to be inhibited (i.e., in renal epithelial cells within an affected area). These liposomes can be produced such that they contain, in addition to monoclonal antibody, other therapeutic agents, such as those described above, which would then be released at the target site.

As used herein, “inhibit” is intended to mean any measurement of a decrease relative to a control. Thus, inhibiting expression of FAT10 means any reduction of FAT10 expression relative to FAT10 expression in a corresponding control. With regard to such inhibition, it is preferable that FAT10 expression can be inhibited by at least about 50%, more preferably at least about 75% up to about 90%. It is expected that FAT10 expression can be substantially precluded in some instances, i.e., having expression reduced by at least about 95%.

Another aspect of the present invention is directed to a method of inhibiting FAT10 protein function in a cell by interfering with FAT10's ability to interact with target proteins.

A further aspect of the present invention is directed to a method of screening for agents effective in treating or preventing renal disease. This method involves providing one or more candidate compounds. The one or more candidate compounds are contacted with a FAT10 gene under conditions effective to express the FAT10 gene in the absence of the one or more candidate compounds. Candidate compounds that inhibit FAT10 expression are identified as agents potentially useful in treating or preventing renal disease.

By use of this method, an agent can be screened for its effectiveness to inhibit FAT10 expression or for efficacy in treating renal diseases or disorders.

According to one embodiment, the screening method is a cell-based assay that utilizes a cell transfected with a DNA construct that includes an isolated nucleic acid molecule encoding FAT10, a reporter gene, and 5′ and 3′ regulatory regions that allow transcription and translation of the nucleic acid molecule encoding the FAT10 protein or polypeptide and the reporter gene. The method also involves incubating a test compound with the transfected cell, measuring the level of the reporter gene expressed in the cell, and comparing the level of expression of the reporter gene expressed in the transgenic cell in the absence of the test compound with the expression of the reporter gene in the presence of the test compound. A decrease in reporter gene expression level indicates that the test compound is able to inhibit FAT10 expression.

In this aspect, a DNA construct is prepared using conventional recombinant technology. Generally, this involves inserting the nucleic acid molecule into an expression system to which the molecule is heterologous (i.e., not normally present). The heterologous nucleic acid molecule is inserted into the expression system or vector in proper sense (5′→3′) orientation relative to the promoter region, and in correct reading frame. The vector contains the necessary elements for the transcription and translation of the inserted protein-coding sequences in a desired host cell or organism.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

A variety of host-vector systems may be utilized to express the protein or RNA-encoding sequences of the present invention. Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used.

Suitable vectors include, but are not limited to, the following viral vectors such as lambda vector system gt11, gt WES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK+/− or KS+/− (see “Stratagene Cloning Systems” Catalog (1993) from Stratagene, La Jolla, Calif., which is hereby incorporated by reference in its entirety), pQE, pIH821, pGEX, pET series (see F. W. Studier et. al., “Use of T7 RNA Polymerase to Direct Expression of Cloned Genes,” Gene Expression Technology Vol. 185 (1990), which is hereby incorporated by reference in its entirety), and any derivatives thereof. Recombinant molecules can be introduced into cells via transformation, particularly transduction, conjugation, mobilization, or electroporation. The DNA sequences are cloned into the vector using standard cloning procedures in the art, as described by Sambrook et al., Molecular Cloning: A Laboratory Manual, Cold Springs Laboratory, Cold Springs Harbor, N.Y. (1989), which is hereby incorporated by reference in its entirety.

Mammalian cells can also be used for any cell-based assays or for production of therapeutic nucleic acids. The mammalian cells can be isolated renal epithelial cells, or they can be commercially available mammalian cell lines. Suitable expression vectors for directing expression in mammalian cells generally include a promoter, as well as other transcription and translation control sequences known in the art. Common promoters operable in mammalian cells include, without limitation, SV40, MMTV, metallothionein-1, adenovirus Ela, CMV, immediate early, immunoglobulin heavy chain promoter and enhancer, and RSV-LTR.

Different genetic signals and processing events control many levels of gene expression (e.g., DNA transcription and messenger RNA (“mRNA”) translation).

Transcription of DNA is dependent upon the presence of a promoter which is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals may not be recognized in or may not function in a prokaryotic system, and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression see Roberts and Lauer, Methods in Enzymology, 68:473 (1979), which is hereby incorporated by reference in its entirety.

Promoters vary in their “strength” (i.e., their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promoter, may also contain any combination of various “strong” transcription and/or translation initiation signals. For instance, efficient translation in E. coli requires a Shine-Dalgarno (“SD”) sequence about 7-9 bases 5′ to the initiation codon (ATG) to provide a ribosome binding site. Thus, any SD-ATG combination that can be utilized by host cell ribosomes may be employed. Such combinations include but are not limited to the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used.

Depending on the vector system and host utilized, any number of suitable transcription and/or translation elements, including constitutive, inducible, and repressible promoters, as well as minimal 5′ promoter elements may be used.

The nucleic acid molecule(s) of the present invention, a promoter molecule (i.e., 5′ regulatory region) of choice, a suitable 3′ regulatory region, and if desired, a reporter gene, are incorporated into a vector-expression system of choice to prepare the nucleic acid construct of present invention using standard cloning procedures known in the art, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety.

In one aspect of the present invention, a reporter gene is included in the DNA construct, and is operably arranged between an isolated nucleic acid molecule encoding a FAT10 protein or polypeptide, and appropriate 5′ and 3′ regulatory regions so that the expression of the FAT10 protein or polypeptide directly correlates to the expression of the reporter gene. Suitable reporter genes for this aspect include GFP, some κ-LacZ, β-gal, and any others known in the art. This construct is suitable for use in one or more of the screening methods described herein.

In one aspect of the present invention, a nucleic acid molecule encoding a FAT10 protein is inserted into a vector in the sense (i.e., 5′→3′) direction, such that the open reading frame is properly oriented for the expression of the encoded protein under the control of a promoter of choice. Single or multiple nucleic acids may be ligated into an appropriate vector in this way, under the control of a suitable promoter, to prepare a nucleic acid construct of the present invention. In another aspect, the nucleic acid molecule may be inserted into the expression system or vector in the antisense (i.e., 3′→5′) orientation.

Once the isolated nucleic acid molecule encoding the FAT10 protein or polypeptide has been cloned into an expression system, it is ready to be incorporated into a host cell. Recombinant molecules can be introduced into cells via transformation, particularly transduction, conjugation, lipofection, protoplast fusion, mobilization, particle bombardment, or electroporation. The DNA sequences are cloned into the host cell using standard cloning procedures known in the art, as described by Sambrook et al., Molecular Cloning: A Laboratory Manual, Second Edition, Cold Springs Laboratory, Cold Springs Harbor, N.Y. (1989), which is hereby incorporated by reference in its entirety. Suitable hosts include, but are not limited to, bacteria, virus, yeast, mammalian cells, insect cells, and the like.

The present invention further pertains to novel agents identified by the above-described screening assays and uses thereof for treatments as described above. Both prophylactic and therapeutic methods of treating a subject at risk of (or susceptible to) a renal disorder or disease condition, or having a disorder or disease condition associated with FAT10 expression or activity, are contemplated.

### EXAMPLES

The examples below are intended to exemplify the practice of the present invention but are by no means intended to limit the scope thereof.

### Example 1

### Tissue Specimens

The HIV-1 transgenic mouse model of HIVAN has been extensively characterized previously. Heterozygous transgenic mice develop proteinuria, progressive renal failure, and histologic renal disease that is identical to HIVAN (Bruggeman et al., “Nephropathy in Human Immunodeficiency Virus-1 Transgenic Mice is Due to Renal Transgene Expression,” J. Clin. Invest. 100:84-92 (1997); Dickie et al., “HIV-Associated Nephropathy in Transgenic Mice Expressing HIV-1 Genes,” Virology 185:109-119 (1991); Kopp et al., “Progressive Glomerulosclerosis and Enhanced Renal Accumulation of Basement Membrane Components in Mice Transgenic for Human Immunodeficiency Virus Type 1 Genes,” Proc. Natl. Acad. Sci. USA 89:1577-1581 (1992), which are hereby incorporated by reference in their entirety). HIVAN biopsy tissue was collected at the time of diagnostic renal biopsy under a protocol approved by the Mount Sinai Institutional Review Board. ADPKD tissue sections were obtained from nephrectomy specimens procured by National Disease Research Interchange and Polycystic Kidney Disease Foundation. All tissues were fixed in 4% paraformaldehye/PBS and embedded in paraffin.

### Example 2

### Generation of the HPT-1 Cell Line

Primary human proximal tubular cells were grown from a HIVAN biopsy specimen by mincing a portion of a fresh cortical renal biopsy sample and plating onto collagen-coated tissue culture plates in media selective for growth of proximal tubular cells according to previously published methods (Wilson et al., “Defined Human Renal Tubular Epithelia in Culture: Growth, Characterization, and Hormonal Response,” Am. J. Physiol. 248:F436-443 (1985), which is hereby incorporated by reference in its entirety). A subpopulation of these cells (subsequently named HPT-1) was conditionally immortalized by infection with VSV-tsTag-IRES-Hygro, a VSV-glycoprotein pseudotyped replication defective lentivirus encoding the SV40 temperature-sensitive large T antigen under the control of the immediate early CMV promoter (FIG. 2A). For all subsequent studies, HPT-1 cells were expanded at 33° C. until they reached 80% confluence and subsequently cultured at 37° C. for 14 days to induce T antigen degradation and cellular differentiation.

### Example 3

### Viral Transduction

To conditionally immortalize PTECs, a lentiviral vector was developed, which encodes the temperature-sensitive SV40 large T-antigen (tsTag) allele tsA58U19 (Racusen et al., “Renal Proximal Tubular Epithelium from Patients with Nephropathic Cystinosis: Immortalized Cell Lines as In Vitro Model Systems,” Kidney Int. 48:536-543 (1995), which is hereby incorporated by reference in its entirety). tsTag was subcloned into pIRES-hyg (Clontech, Mountain View, Calif.). A fragment including the CMV promoter tsTag and hygromycin-resistance genes was subsequently subcloned into pHR-CMV-IRES2-EGFP-ΔB (gift of Dr. James C. Mulloy, Memorial Sloan Kettering Cancer Center), resulting in the vector pHR′-tsTAg-IRES-Hygro (FIG. 2A).

pNL4-3:ΔG/P-EGFP, a gag/pol-deleted HIV-1 construct containing EGFP in the gag open reading frame (FIG. 2B) (Husain et al., “HIV-1 Nef Induces Proliferation and Anchorage-Independent Growth in Podocytes,” J. Am. Soc. Nephrol. 13:1806-1815 (2002), which is hereby incorporated by reference in its entirety) and pHR′-IRES-EGFP (EGFP control) (FIG. 2C) was used to generate VSV-G-pseudotyped virus for infection of HPT-1 cells as previously described (Husain et al., “HIV-1 Nef Induces Proliferation and Anchorage-Independent Growth in Podocytes,” J. Am. Soc. Nephrol. 13:1806-1815 (2002), which is hereby incorporated by reference in its entirety). A multiplicity of infection (“MOI”) of 0.5 was used to infect HPT-1 cells, because higher MOIs resulted in excessive cytotoxicity at 3 and 7 days post infection.

### Example 4

### Characterization of HPT-1 Cells

HPT-1 cells were grown and differentiated on glass coverslips that had been coated with type-1 rat tail collagen (BD Biosciences, San Jose, Calif.). Cells were fixed in 4% paraformaldehyde/PBS. Endogenous alkaline phosphatase activity was detected by incubating cells in 10 mM Tris pH 9.0 with 0.3 mM 5-bromo, 4-chloro, 3-indolylphosphate and nitroblue tetrazolium (Roche, Indianapolis, Ind.). For immunocytochemical detection of aquaporin-1 and cytokeratin, HPT-1 cells were permeablized with 0.1% Triton® X-100. Primary antibodies used to characterize HPT-1 cells included anti-aquaporin-1 (AQP11-S), anti-aquaporin-2 (AQP21-A) (Alpha Diagnostics, San Antonio, Tex.), anti-cytokeratin (BT-571, BTI, Stoughton, Mass.) anti-vimentin (V2258, Sigma, St. Louis, Mo.), anti-smooth muscle myosin (MAB3568, Chemicon, Temecula, Calif.), and Tamm-Horsfall protein (55140, Cappel Laboratories, Westchester, Pa.). Primary antibodies were detected using Cy3-labelled goat anti-rabbit IgG or Cy3-labeled goat anti-mouse IgG (KPL, Gaithersburg, Mass.) diluted 1:100.

### Example 5

### Analysis of FAT10 RNA Expression in HPT-1 Cells and Kidneys from HIV-1-Transgenic Mice

Total RNA was harvested from HPT-1 cells after infection with VSV-NL4-3:ΔG/P-EGFP or VSV-HR′-IRES-EGFP from kidneys from adult transgenic mice with severe histologic renal disease and proteinuria, transgenic mice without significant histologic renal disease, and normal mice. 10 μg of RNA from each sample was resolved on 1.2% agarose/formaldehyde gel and transferred to a 0.45-μm Biodyne membrane (Pall Corp, East Hills, N.Y.).

For analysis of FAT10 expression in HPT-1 cells, a portion of the second exon of human FAT10 was amplified by PCR using cDNA from VSV-NL4-3:ΔG/P-EGFP-infected HPT-1 cells using HotStar Master Mix kit (Qiagen, Valencia, Calif.) and the following primers:

| | |
|---|---|
| 5′-GATCTTAAAGCCACGGAGAA-3′ | (SEQ ID NO: 5) |
| (sense) | |
| and | |
| 5′-CATCCCACCCAAATCTTACT-3′ | (SEQ ID NO: 6) |
| (antisense) | |

with the following thermocycler parameters: 1 min at 94° C., 1 min at 58° C., 1 min at 72° C. for 40 cycles.

For analysis of FAT10 expression in murine kidneys, the murine FAT10 coding sequence was amplified using cDNA from HIV-transgenic kidneys using primers:

(SEQ ID NO: 7)

5′-CGGGATCCCACCATGGCTTCTGTCCGCACCTGTG-3′
(sense)
and

(SEQ ID NO: 8)

5′-GGAATTCGGTTATCCCCCAGTGCAGTGTGTTGTC-3′
(antisense)

and the following thermocycler parameters: 15 min at 95° C. for 1 cycle, 30 sec at 94° C., 1 min at 58° C., 1 min at 72° C. for 4 cycles, 30 sec at 94° C., 1 min at 64° C., and 1 min at 72° C. for 31 cycles.

PCR products were purified using Qiaquick columns (Qiagen) and labeled with dCTP³² (Perkin-Elmer, Wellesley, Mass.) using Ready to Go dCTP Labeling Beads (Amersham Biosciences, Piscataway, N.J.) according to the manufacturer's protocol. GAPDH and ribosomal RNA bands were analyzed to ensure equal loading of RNA.

### Example 6

### Generation of FAT10 Expression Vectors

The FAT10 coding sequence was amplified from plasmid MGC-21200 (ATCC) using the following primers:

(SEQ ID NO: 9)

5′-CGGGATCCACCATGGCTCCCAATGCTTCCTGCC-3′
(sense, BamHI adapter underlined)
and

(SEQ ID NO: 10)

5′-GGAATTCGGTTACCCTCCAATACAATAAGATGCCAG-3′
(antisense, EcoRI adapter underlined)

and the following thermocycler parameters: 15 min at 95° C. for 1 cycle, 30 sec at 94° C., 1 min at 58° C., 1 min at 72° C. for 4 cycles, 30 sec at 94° C., 1 min at 64° C., and 1 min at 72° C. for 31 cycles. The PCR product was cloned into VVPW/BE (see below) and pcDNA4/HisMaxC (Invitrogen) using BamH1 and EcoR1 (New England Biolabs, Beverly, Mass.), resulting in the pVVPW/BE-FAT10 (FIG. 2D) and pcDNA4-FAT10 (FIG. 2E) vectors. pVVPW/BE-FAT10 expresses FAT10 in a biscistronic transcript with EGFP expressed as a separate protein, whereas pcDNA4-FAT10 expresses FAT10 with an N-terminal Xpress epitope tag.

### Example 7

### Generation of the Plasmid VVPW/BE

The lentiviral vector VVPW/BE was derived by the insertion of the BE (BiP/EGFP) cassette containing a internal ribosome entry site from the human heat shock protein BiP upstream of the EGFP gene into VVPW (Fedorova et al., “Lentiviral Gene Delivery to CNS by Spinal Intrathecal Administration to Neonatal Mice,” J. Gene Med., 8:414-424 (2006), which is hereby incorporated by reference in its entirety). To generate this cassette, the NotI site at the 3′ end of the EGFP reporter gene in pHR′PGK/EGFP vector (Gusella et al., “In Vivo Gene Transfer to Kidney by Lentiviral Vector,” Kidney Int. 61:32-36 (2002), which is hereby incorporated by reference in its entirety) was first removed following Notl digestion, fill-in with Klenow polymerase, and self-ligation. In parallel, a NcoI linker was introduced in place of the KpnI site at the 3′ of BiP in ZQ-30-1-S-IRES plasmid. The BiP element, digested with NotI and NcoI, and the EGFP reporter gene, excised with NcoI and XhoI, were then introduced between the NotI and XhoI sites of VVPW in a triple ligation to generate VVPW/BE.

### Example 8

### Generation of Short Hairpin RNA Vectors

Two Short Hairpin RNA (“shRNA”) vectors were constructed to knockdown expression of FAT10. One vector (pVIRHD/E-FAT10.1) is targeted against the first exon of FAT10 and the other (pVIRHD/E-FAT10.2) against the second exon. The VIRHD/E/siLuc lentivector expressing the anti-luciferase siRNA was used as negative control. Each vector was constructed by annealing complementary 61-mer oligonucleotides containing the 19 nucleotide sequence corresponding to the target FAT10 sequence in the sense and antisense orientation separated by a 9 nucleotide spacer region, allowing generation of a shRNA. Oligonucleotide sequences are provided in Table 1. Annealed oligonucleotides were inserted under the control of the human H1 promoter in the self-inactivating pVIRHD/E lentiviral vector (FIG. 2F) derived from VVPW/EGFP (Fedorova et al., “Lentiviral Gene Delivery to CNS by Spinal Intrathecal Administration to Neonatal Mice,” J. Gene Med., 8:414-424 (2006), which is hereby incorporated by reference in its entirety). The resulting lentiviral vectors, which also express the reporter EGFP from the constitutive murine phosphoglycerate kinase promoter, were packaged into VSV-G pseudotyped virions using the methods described above.

TABLE 1
Oligonucleotides Used for Generating shRNA Vectors

pVIRHD/E-FAT10.1

| | |
|---|---|
| sense | 5′-GATCCTGCTTCCTGCCTCTGTGTGTTGATATCCGCA |
| | CACAGAGGCAGGAAGCATTTTTTGG-3′ |
| | (SEQ ID NO: 11) |
| anti-sense | 5′-AATTCCAAAAAATGCTTCCTGCCTCTGTGTGCGGAT |
| | ATCAACACACAGAGGCAGGAAGCAG-3′ |
| | (SEQ ID NO: 12) |

pVIRHD/E-FAT10.2

| | |
|---|---|
| sense | 5′-GATCCGAACATGTCCGGTCTAAGATTGATATCCGT |
| | CTTAGACCGGACATGTTCTTTTTTGG-3′ |
| | (SEQ ID NO: 13) |
| anti-sense | 5′-AATTCCAAAAAAGAACATGTCCGGTCTAAGACGGA |
| | TATCAATCTTAGACCGGACATGTTCG-3′ |
| | (SEQ ID NO: 14) |
| pVIRHD/E/ | 5′-GATCCGTGCGTTGCTAGTACCAACTTCAAGAGAGT |
| siLuc | TGGTACTAGCAACGCACTTTTTTGG-3′ |
| | (SEQ ID NO: 15) |
| sense | 5′-AATTCCAAAAAAGTGCGTTGCTAGTACCAACTCTC |
| | TTGAAGTTGGTACTAGCAACGCACG-3′ |
| | (SEQ ID NO: 16) |
| anti-sense | 5′-GATCCTGCTTCGTGCCTCTGTGTGTTGATATCCGCA |
| | CACAGAGGCAGGAAGCATTTTTTGG-3′ |
| | (SEQ ID NO: 17) |

### Example 9

### Generation of Anti-FAT10 Antiserum

Rabbit polyclonal antiserum against FAT10 was raised against the peptide APNASCLCVHVRSE (SEQ ID NO:3), which spans the splice junction between the first and second exons of FAT10. This peptide was conjugated to Keyhole Limpet Hemocyanin prior to inoculation. Peptide synthesis, inoculations, and harvesting of antiserum were performed by Open Biosystems (Huntsville, Ala.). Antiserum used in these studies was collected 70 days post inoculation.

### Example 10

### Immunohistochemistry

Paraformaldhyde-fixed paraffin-embedded tissue was stained for FAT10 using FAT10 antiserum at a 1:100 dilution followed by incubation with biotinylated rabbit anti-goat IgG at a 1:200 dilution (KPL, Gaithersburg, Mass.). The Vector ABC Elite Kit and aminoethylcarbazole (Vector Labs, Burlingame, Calif.) were used according to previously published protocol (Ross et al., “Microcyst Formation and HIV-1 Gene Expression Occur in Multiple Nephron Segments in HIV-Associated Nephropathy,” J. Am. Soc. Nephrol. 12:2645-2651 (2001), which is hereby incorporated by reference in its entirety). In order to ensure specificity of the antiserum, staining controls included using preimmune serum from the same rabbit used to generate the antiserum and incubating the antiserum with 10 μg/mL of the immunizing peptide for 60 minutes prior to use in immunohistochemistry.

### Example 11

### FAT10 shRNA Inhibition Studies

Separate populations of HPT-1 cells were transduced with VSV-pseudotyped VIRHD/e-FAT10.1 and VIRHD/E-FAT10.2 at an MOI of 10. After expansion and differentiation, 10⁷ cells were transfected with pcDNA4-FAT10 (FIG. 2E) using Lipofectamine 2000 (Invitrogen). Lysate from pcDNA4-FAT10-transfected HPT-1 cells was used for detection of Xpress-tagged FAT10 by western blotting using Anti-Xpress-HRP (Invitrogen) diluted 1:2000.

### Example 12

### Flow Cytometry

Differentiated HPT-1 cells were transfected with VVPW/BE-FAT10 or VVPW/BE (control) using Lipofectamine 2000 (Invitrogen) using a DNA:lipid ratio of 1:3 according to the manufacturer's protocol. 24 hours after transfection, 1.2×10⁶ HPT-1 cells were stained using the Vybrant apoptosis #6 kit (Invitrogen) according to the manufacturer's protocol. Cells were then analyzed by flow cytometry at the Mount Sinai Flow Cytometry Shared Research Facility using a FACSVantage flow cytometer (BD Biosciences). Transfected cells were identified by the presence of EGFP fluorescence. Apoptotic cells were identified by the presence of annexin V staining in the absence of propidium staining and necrotic cells were identified by the presence of propidium staining. Chi-square testing was used to compare the number of apoptotic and apoptotic plus necrotic cells transfected with VVPW/BE-FAT10 to cells transfected with VVPW/BE. A 2-sided p value of <0.05 was used to define statistical significance.

To study the effect of FAT10 inhibition upon HIV-induced apoptosis, separate populations of HPT-1 cells that had been stably transduced with VIRHD/c-FAT 10.1, VIRHD/E-FAT10.2, or VTRHD/E/siLuc were infected with VSV-NL4-3:ΔG/P-EGFP as described above. Seven days after infection, apoptosis in each population was analyzed as described above.

### Example 13

### Development of a Tubular Epithelial Cell Line, HPT-1, from a Patient with HIVAN

Host genetic factors strongly influence susceptibility to HIVAN. Therefore, to maximize the relevance of the studies described herein to HIVAN pathogenesis, a PTEC line from a patient with HIVAN was developed for use. PTECs were chosen because they are the predominant epithelial cell type in the kidney, they have been shown to be infected by HIV-1, and they are important in disease progression in HIVAN (Ross et al., “Microcyst Formation and HIV-1 Gene Expression Occur in Multiple Nephron Segments in HIV-Associated Nephropathy,” J. Am. Soc. Nephrol. 12:2645-2651 (2001), which is hereby incorporated by reference in its entirety).

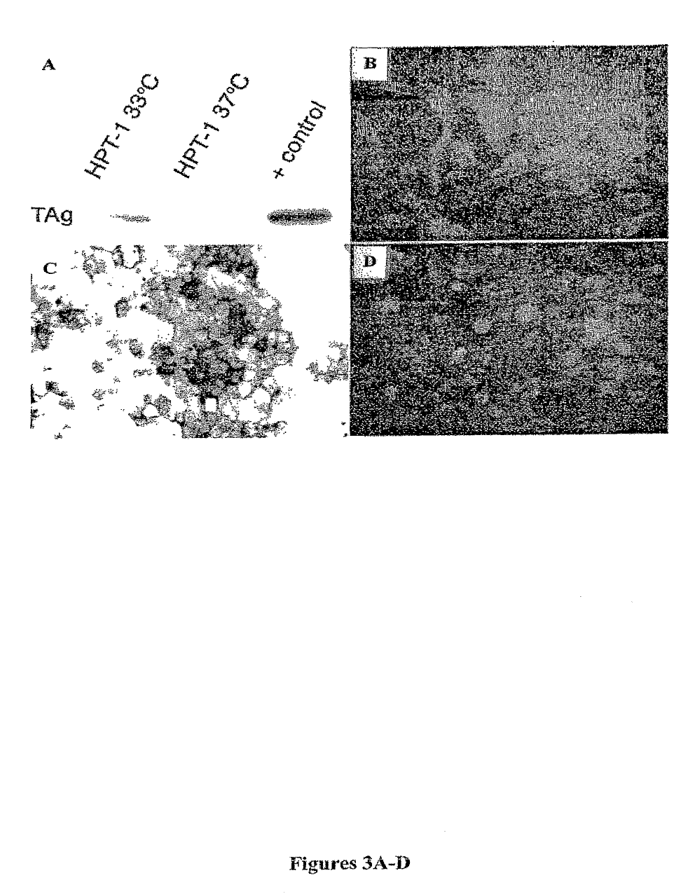

Primary PTECs grown from a HIVAN biopsy were conditionally immortalized by stable transduction with a lentivirus encoding the SV40 temperature-sensitive large T antigen under the control of the CMV promoter (FIG. 2A). The resulting cell population (subsequently referred to as HPT-1) grows indefinitely under permissive conditions (33° C.), but at 37° C. T antigen degrades (FIG. 3A) and the cells are induced to differentiate maximally. HPT-1 cells express PTEC markers such as cytokeratin, alkaline phosphatase, and aquaporin-1 (FIGS. 3B, 3C, and 3D) HPT-1 cells do not express nonepithelial proteins such as vimentin or smooth muscle myosin, or Tamm Horsfall protein or aquaporin-2 (markers for thick ascending limb of Henle and collecting duct, respectively). HPT-1 cells are not latently infected by HIV-1 as determined by PCR analysis.

### Example 14

### FAT10 mRNA Expression Following in Vitro HIV-1 Transduction of Human Proximal Tubular Cells

In preliminary studies using oligonucleotide microarrays, it was found that FAT10 was one of the most robustly upregulated genes in HIV-infected HPT-1 cells. To determine the specificity of the increased expression of FAT10, northern blotting was performed using RNA from HPT-1 cells infected with VSV-NL4-3:ΔG/P-EGFP (FIG. 2B), a gag/pol-deleted virus derived from the same proviral construct used to create the HIV-1 transgenic HIVAN model (Dickie et al., “HIV-Associated Nephropathy in Transgenic Mice Expressing HIV-1 Genes,” Virology 185:109-119 (1991); Husain et al., “HIV-1 Nef Induces Proliferation and Anchorage-Independent Growth in Podocytes,” J. Am. Soc. Nephrol. 13:1806-1815 (2002), which are hereby incorporated by reference in their entirety). VSV-HR′-IRES-EGFP, a lentivirus expressing enhanced green fluorescent protein (FIG. 2C) was used as a control. Since infection of RTECs in vitro by HIV-1 is inefficient (Conaldi et al., “HIV-1 Kills Renal Tubular Epithelial Cells In Vitro by Triggering an Apoptotic Pathway Involving Caspase Activation and Fas Upregulation,” J. Clin. Invest. 102:2041-2049 (1998); Ray et al., “Infection of Human Primary Renal Epithelial Cells with HIV-1 from Children with HIV-Associated Nephropathy,” Kidney Int. 53:1217-1229 (1998), which are hereby incorporated by reference in their entirety), both viruses were pseudotyped with the vesicular stomatitis virus glycoprotein (VSV-G) to maximize the efficiency of infection. FAT10 expression was markedly increased in HPT-1 cells infected with VSV-pNL4-3:ΔG/P-EGFP at 3 and 7 days post infection, but not at 12 and 24 hours post infection (FIG. 4A). FAT10 was not expressed in control-infected cells or non-infected cells at any time point (FIG. 4A).

### Example 15

### FAT10 RNA Expression in Kidneys from HIV-1 Transgenic Mice

FAT10 expression in kidneys from the HIV-1 transgenic model of HIVAN was studied. These mice develop proteinuria, renal failure, and histologic renal disease that is identical to HIVAN (Kopp et al., “Progressive Glomerulosclerosis and Enhanced Renal Accumulation of Basement Membrane Components in Mice Transgenic for Human Immunodeficiency Virus Type 1 Genes,” Proc. Natl. Acad. Sci. USA 89:1577-1581 (1992), which is hereby incorporated by reference in its entirety). Kidneys were obtained from transgenic mice with severe proteinuria and histologic disease, transgenic mice without overt proteinuria or histologic disease, and normal adult mice. FAT10 expression was markedly increased in kidneys from HIV-transgenic mice with severe renal disease, with lesser FAT0 levels detectable in kidneys from HIV-transgenic mice without overt renal disease (FIG. 4B). FAT10 expression was minimally detectable in kidneys from normal adult mice (FIG. 4B).

### Example 16

### FAT10 Expression Induces Apoptosis in HPT-1 Cells

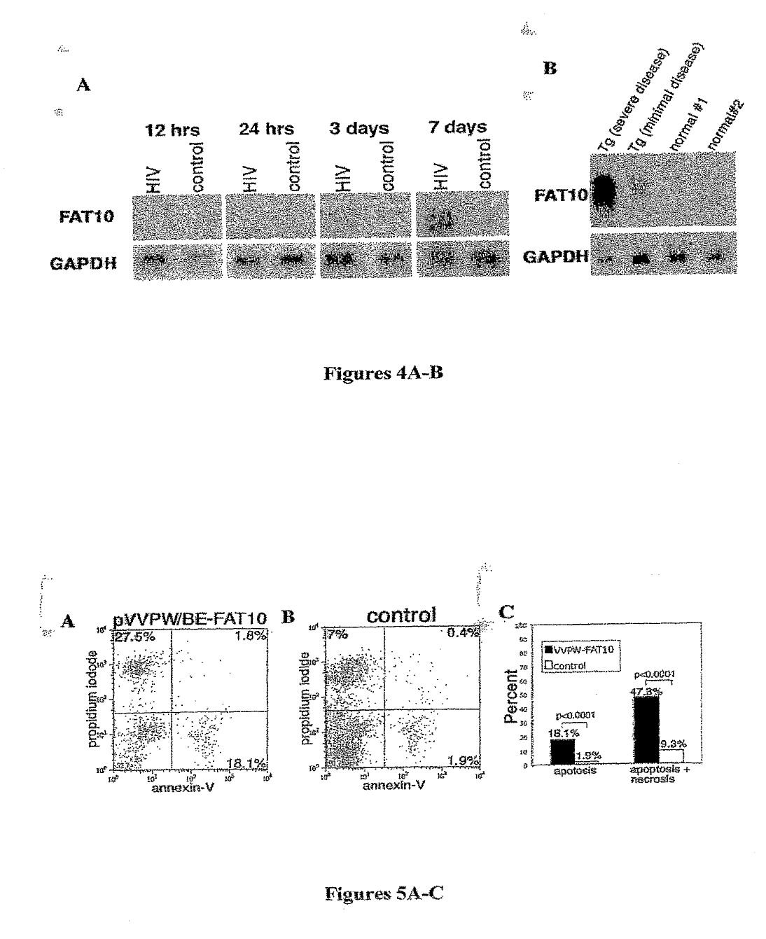

Since FAT10 expression and epithelial apoptosis are increased in HIVAN, it was studied whether FAT10 is capable of inducing apoptosis in HPT-1 cells. HPT-1 cells were transfected with a plasmid encoding FAT10 and EGFP in a dicistronic vector (pVVPW/BE-FAT10, FIG. 2D), allowing identification of transfected cells by EGFP fluorescence. 24 hours after transfection with pVVPW/BE-FAT10, 18.1% of EGFP-expressing cells were apoptotic as determined by annexin V staining (FIGS. 5A and 5C), whereas only 1.9% of HPT-1 cells transfected with the control vector (VVPW/BE) were apoptotic (FIGS. 5B and 5C). Moreover, 47.3% of pVVPW/BE-FAT10 transfected cells were either apoptotic or necrotic (positive for propidium and/or annexin V staining), but only 9.3% of vector-transfected cells were apoptotic or necrotic (FIG. 5C). Levels of apoptosis and apoptosis plus necrosis were significantly greater in the cells expressing FAT10 (p<0.0001).

### Example 17

### Inhibition of FAT10 Prevents HIV-Induced Apoptosis of RTECs

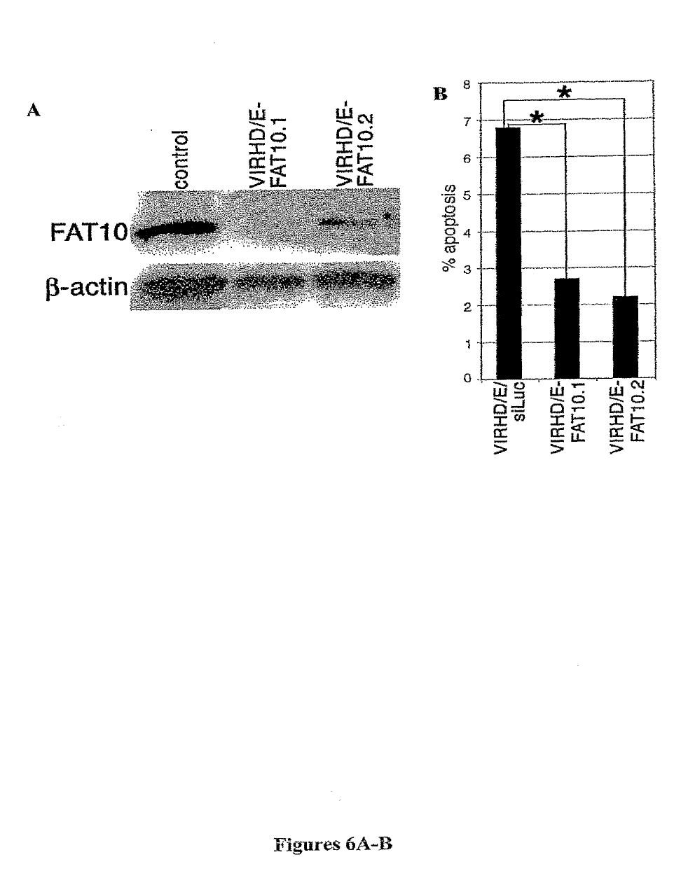

To determine if FAT10 has a direct causal role in HIV-induced apoptosis of RTECs, it was studied whether specific inhibition of endogenous FAT10 using shRNA vectors could prevent apoptosis of RTECs after infection with HIV-1. Two lentiviral vectors were generated (VIRHD/e-FAT10.1 and VIRHD/E-FAT10.2) to stably transduce HPT-1 cells with shRNA directed against two separate regions of the FAT10 transcript. To determine the efficacy of these vectors in suppressing FAT10 protein expression, HPT-1 cells wore stably transduced with the shRNA vectors and were subsequently transfected with the FAT10 expression vector pcDNA4-FAT10. Both shRNA vectors effectively inhibited expression of FAT10 protein (FIG. 6A).

It was then determined whether preventing upregulation of endogenous FAT10 could prevent apoptosis in HPT-1 cells after infection by HIV-1. HPT-1 cells that had been stably transduced were infected with VIRHD/e-FAT10.1, VIRHD/E-FAT10.2, or VIRHD/E/siLuc with VSV-NL4-3:ΔG/P-EGFP. Cells that expressed either anti-FAT10 shRNA construct demonstrated significantly lower levels of apoptosis as compared to cells expressing the shRNA control vector VIRHD/E/siLuc (FIG. 6B, p<0.0001).

### Example 18

### FAT10 Protein Expression is Increased in Kidneys from HIV-1 Transgenic Mice

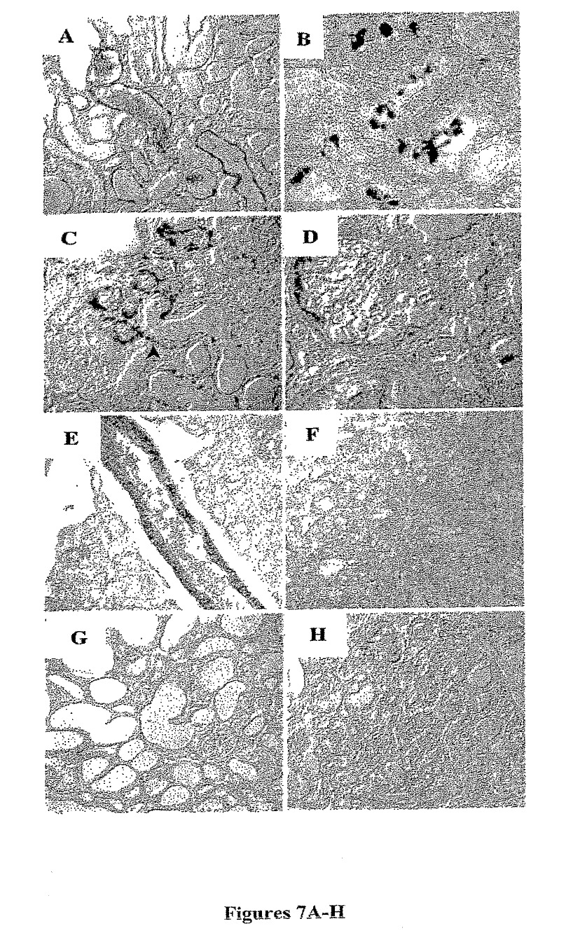

FAT10 antiserum was used to perform immunohistochemical analysis of FAT10 expression and localization in kidneys from HIV-1 transgenic mice and in normal controls. FAT10 staining was focal with expression in RTECs in some nephrons (FIG. 7A), sloughing (often apoptotic) epithelial cells (FIG. 7B), and in cells just beneath the epithelial layer (FIG. 7C). Focal expression of FAT10 was also present in glomeruli (FIG. 7D) and in vascular smooth muscle cells (FIG. 7E). In kidneys from normal adult mice, FAT10 was not detected in glomeruli, tubules, or interstitium (FIG. 7F), but was detected in vascular smooth muscle cells. Preimmune serum controls were negative (FIG. 7G) and preincubation of the antiserum with the peptide used to generate the antiserum abolished all staining (FIG. 7H). Immunostaining with other polyclonal antibodies to FAT10 (Lee et al., “Expression of the FAT10 Gene is Highly Upregulated in Hepatocellular Carcinoma and Other Gastrointestinal and Gynecological Cancers,” Oncogene 22:2592-2603 (2003); Liu et al., “A MHC-Encoded Ubiquitin-Like Protein (FAT10) Binds Noncovalently to the Spindle Assembly Checkpoint Protein MAD2,” Proc. Natl. Acad. Sci. USA 96:4313-4318 (1999), which are hereby incorporated by reference in their entirety) resulted in similar staining.

### Example 19

### FAT10 Expression in HIVAN and ADPKD

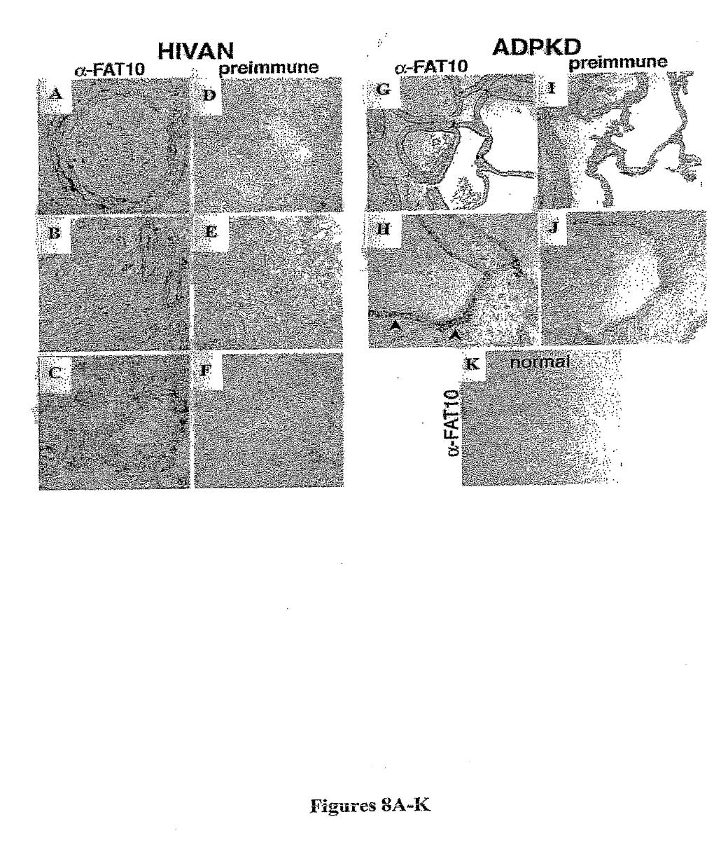

To determine whether FAT10 expression is increased in patients with HIVAN, immunohistochemistry was performed using HIVAN biopsy specimens. FAT10 was expressed in areas of periglomerular fibrosis surrounding Bowman's capsule (FIG. 8A), in tubular epithelial cells (FIG. 8B), and in arterial smooth muscle cells (FIG. 8C). Preimmune serum controls using serial sections were negative (FIGS. 8D, E, F).

Since ADPKD, like HIVAN, is characterized by tubular cystic dilatation and dysregulated RTEC apoptosis, immunohistochemistry was performed to investigate whether FAT10 is expressed in kidneys from patients with ADPKD. FAT10 was strongly expressed in RTECs lining cysts (FIG. 8G-J), particularly in areas where the epithelium had become hyperplastic (FIG. 8H). Preimmune controls were negative (FIGS. 8I and J) and FAT10 expression was not detected in normal adult human kidneys (FIG. 8K).

HIVAN and ADPKD are important causes of renal disease. HIVAN is the third-leading cause of end stage renal disease (“ESRD”) in African Americans aged 20-64 and is the most common cause of chronic kidney failure in HIV-1 infected patients (Ross et al., “HIV-Associated Nephropathy,” Aids 18:1089-1099 (2004), which is hereby incorporated by reference in its entirety). ADPKD is the most common lethal inherited disease in humans, occurring in 1 in 800 live births and affecting 4-6 million people worldwide (Wilson, “Polycystic Kidney Disease,” N. Engl. J. Med. 350:151-164 (2004), which is hereby incorporated by reference in its entirety).

HIVAN is caused by expression of HIV-1 genes in infected renal epithelial cells (Bruggeman et al., “Nephropathy in Human Immunodeficiency Virus-1 Transgenic Mice is Due to Renal Transgene Expression,” J. Clin. Invest. 100:84-92 (1997); Bruggeman et al., “Renal Epithelium is a Previously Unrecognized Site of HIV-1 Infection,” J. Am. Soc. Nephron. 11:2079-2087 (2000); Marras et al., “Replication and Compartmentalization of HIV-1 in Kidney Epithelium of Patients with HIV-Associated Nephropathy,” Nat. Med. 8:522-526 (2002); Winston et al., “Nephropathy and Establishment of a Renal Reservoir of HIV Type 1 During Primary Infection,” N. Engl. J. Med. 344:1979-1984 (2001), which are hereby incorporated by reference in their entirety). Host genetic factors are important in modulating the predisposition to developing HIVAN in patients and in HIV-1 transgenic mice (Freedman et al., “Familial Clustering of End-Stage Renal Disease in Blacks with HIV-Associated Nephropathy,” Am. J. Kidney Dis. 34:254-258 (1999); Gharavi et al., “Mapping a Locus for Susceptibility to HIV-1-Associated Nephropathy to Mouse Chromosome 3,” Proc. Natl. Acad. Sci. USA 101:2488-2493 (2004); Gharavi et al., “Genetic Background Modifies the Development of Renal Disease in HIV-1 Transgenic Mice,” J. Amer. Soc. Nephrol. 10:404A (1999); Abbott et al., “Human Immunodeficiency Virus/Acquired Immunodeficiency Syndrome-Associated Nephropathy at End-Stage Renal Disease in the United States: Patient Characteristics and Survival in the Pre Highly Active Antiretroviral Therapy Era,” J. Nephrol. 14:377-383 (2001), which are hereby incorporated by reference in their entirety). Most previous studies of the response of renal epithelial cells to HIV-1 infection have used cell lines from rodents or hosts that are not susceptible to developing HIVAN (Caucasians). In these studies, RTECs from a patient with HIVAN were used to maximize the likelihood that the in vitro cellular response to infection will be relevant to HIVAN pathogenesis.

Ubiquitin-like proteins (“UBL”) have important roles in the pathogenesis of several diseases, including malignancy, neurodegenerative diseases, immune disorders, and infectious diseases (Ciechanover et al., “The Ubiquitin System: From Basic Mechanisms to the Patient Bed,” IUBMB Life 56:193-201 (2004), which is hereby incorporated by reference in its entirety). FAT10 is a 165 amino acid UBL containing two ubiquitin-like domains, each of which is approximately one-third identical to ubiquitin (Raasi et al., “The Ubiquitin-Like Protein FAT10 Forms Covalent Conjugates and Induces Apoptosis,” J. Biol. Chem. 276:35334-35343 (2001), which is hereby incorporated by reference in its entirety). FAT10 was first cloned by Fan et al., who discovered the gene by cDNA hybridization selection while analyzing the human HLA-F locus for novel genes (Fan et al., “Identification of Seven New Human MHC Class I Region Genes around the HLA-F Locus,” Immunogenetics 44:97-103 (1996), which is hereby incorporated by reference in its entirety). FAT10 was initially reported to be expressed in mature B-lymphocytes and dendritic cells, suggesting a role for the gene in antigen processing/presentation and immune response (Bates et al., “Identification and Analysis of a Novel Member of the Ubiquitin Family Expressed in Dendritic Cells and Mature B Cells,” Eur. J. Immunol. 27:2471-2477 (1997), which is hereby incorporated by reference in its entirety). Other investigators have found, however, that a variety of transformed cell lines express FAT10 when they are exposed to tumor necrosis factor-alpha (TNF-α) and interferon gamma (IFN-γ), but not IFN-α (Raasi et al., “The Ubiquitin-Like Protein FAT 0 Forms Covalent Conjugates and Induces Apoptosis,”J. Biol. Chem. 276:35334-35343 (2001), which is hereby incorporated by reference in its entirety). This is in contrast to the ubiquitin-like protein ISG-15, which is induced by IFN-α and not IFN-γ (Ritchie et al., “ISG15: The Immunological Kin of Ubiquitin,” Semin. Cell. Dev. Biol. 15:237-246 (2004), which is hereby incorporated by reference in its entirety). Although both the cytokine-mediated induction of FAT10 is vitro and its genomic localization suggest FAT10 may have a role in immunity, the in vivo role of FAT10 remains obscure.

The first evidence that FAT10 is a proapoptotic molecule came from Rassi and colleagues who reported that attempts to produce cell lines that would constitutively express FAT10 protein were unsuccessful due to increased cell death (Raasi et al., “A Ubiquitin-Like Protein which is Synergistically Inducible by Interferon-Gamma and Tumor Necrosis Factor-Alpha,” Eur. J. Immunol. 29:4030-4036 (1999), which is hereby incorporated by reference in its entirety). The same group later found that inducible expression of FAT10 in mouse fibroblasts results in caspase-dependent apoptosis that is abrogated by deletion of the c-terminal diglycine motif of FAT10 (Raasi et al., “The Ubiquitin-Like Protein FAT10 Forms Covalent Conjugates and Induces Apoptosis,” J. Biol. Chem. 276:35334-35343 (2001), which is hereby incorporated by reference in its entirety). Since this motif is necessary for covalent attachment of ubiquitin and UBLs to target proteins, this study suggested that FAT10 must become covalently attached to as-yet unidentified protein(s) in order to induce apoptosis. Further supporting a role for FAT10 in apoptosis, Coyle et al. reported that induction of apoptosis in hepatoma cells by exposure to transforming growth factor-beta (“TGF-β”) results in strong upregulation of FAT10 (Coyle et al., “Characterization of the Transforming Growth Factor-Beta 1-Induced Apoptotic Transcriptome in FaO Hepatoma Cells,” J. Biol. Chem. 278:5920-5928 (2003), which is hereby incorporated by reference in its entirety). Since TGF-β is upregulated in renal specimens from patients with HIVAN (Yamamoto et al., “Increased Levels of Transforming Growth Factor-Beta in HIV-Associated Nephropathy,” Kidney Int. 55:579-592 (1999), which is hereby incorporated by reference in its entirety) and has been implicated as a mediator in the pathogenesis of many forms of progressive renal failure, FAT10 may have be involved in TGF-β-induced apoptosis.

FAT10 conjugation to proteins has recently been shown to target proteins for ubiquitin-independent proteasomal degradation (Hipp et al., “FAT10, A Ubiquitin-Independent Signal for Proteasomal Degradation,” Mol. Cell. Biol. 25:3483-3491 (2005), which is hereby incorporated by reference in its entirety). While the proteins to which FAT10 becomes covalently conjugated remain unknown, FAT10 has been shown to non-covalently interact with two proteins, MAD2 (Liu et al., “A MHC-Encoded Ubiquitin-Like Protein (FAT10) Binds Noncovalently to the Spindle Assembly Checkpoint Protein MAD2,” Proc. Natl. Acad. Sci. USA 96:433-4318 (1999), which is hereby incorporated by reference in its entirety) and NEDD8 ultimate buster 1L (“NUB1L”) (Hipp et al., “NEDD8 Ultimate Buster-1L Interacts with the Ubiquitin-Like Protein FAT10 and Accelerates its Degradation,” J. Biol. Chem. 279:16503-16510 (2004), which is hereby incorporated by reference in its entirety). MAD2 is a mitotic spindle assembly checkpoint protein and its inhibition can lead to genomic instability and subsequent apoptosis (Kops et al., “Lethality to Human Cancer Cells through Massive Chromosome Loss by Inhibition of the Mitotic Checkpoint,” Proc. Natl. Acad. Sci. USA 101:8699-8704 (2004), which is hereby incorporated by reference in its entirety). NUB1L has been reported to bind non-covalently to another UBL, NEDD8. NUB1L shortens the half-life of NEDD8 by facilitating its degradation via the proteasome (Tanaka et al., “Regulation of the NEDD8 Conjugation System by a Splicing Variant, NUB1L,” J. Biol. Chem. 278:32905-32913 (2003), which is hereby incorporated by reference in its entirety). Since NEDD8 modifies the function of several proteins, including ubiquitin ligases (Parry et al., “Regulation of Cullin-Based Ubiquitin Ligases by the Nedd8/RUB Ubiquitin-Like Proteins,” Semin. Cell. Dev. Biol. 15:221-229 (2004), which is hereby incorporated by reference in its entirety) and p53 (Xirodimas et al., “Mdm2-Mediated NEDD8 Conjugation of p53 Inhibits its Transcriptional Activity,” Cell 118:83-97 (2004), which is hereby incorporated by reference in its entirety), dysregulated FAT10 expression could lead to apoptosis by altering intracellular levels of NEDD8. Thus, increased apoptosis caused by increased FAT10 expression may result from covalent and/or non-covalent interactions of FAT 0 with several cellular proteins.

Ubiquitin and UBLs are important cofactors in the HIV-1 life cycle. HIV-1 Tat, for example, must be ubiquitylated to maximally transactivate transcription from the HIV-1 LTR promoter (Bres et al., “A Non-Proteolytic Role for Ubiquitin in Tat-Mediated Transactivation of the HIV-1 Promoter,” Nat. Cell Biol. 5:754-761 (2003), which is hereby incorporated by reference in its entirety) and the Gag late protein p6 must be ubiquitylated to allow budding of HIV-1 virions from the cell membrane (Garrus et al., “Tsg101 and the Vacuolar Protein Sorting Pathway are Essential for HIV-1 Budding,” Cell 107:55-65 (2001), which is hereby incorporated by reference in its entirety). p6 has also recently been shown to be capable of modification by the UBL SUMO-1 (Gurer et at., “Covalent Modification of Human Immunodeficiency Virus Type 1 p6 by SUMO-1,” J. Virol. 79:910-917 (2005), which is hereby incorporated by reference in its entirety). Other HIV-1 proteins such as Vif and Vpu induce ubiquitylation and subsequent degradation of host proteins (Sheehy et al., “The Antiretroviral Enzyme APOBEC3G is Degraded by the Proteasome in Response to HIV-1 Vif,” Nat. Med. 9:1404-1407 (2003); Yu et al., “Induction of APOBEC3G Ubiquitination and Degradation by an HIV-1 Vif-Cul5-SCF Complex,” Science 302:1056-1060 (2003); Schubert et al., “CD4 Glycoprotein Degradation Induced by Human Immunodeficiency Virus Type 1 Vpu Protein Requires the Function of Proteasomes and the Ubiquitin-Conjugating Pathway,” J. Virol. 72:2280-2288 (1998), which are hereby incorporated by reference in their entirety). Whether HIV-1 proteins are targets of FAT10 conjugation or induce FAT10 conjugation to host proteins is currently under investigation.

These studies demonstrate that HIV-1 expression in RTECs induces expression of FAT10 and that FAT10 expression induces apoptosis in RTECs. Moreover, inhibition of FAT10 expression results in suppression of HIV-induced apoptosis of RTECs. Since FAT10 expression is increased in HIVAN and ADPKD, it plays an important role in the dysregulation of apoptosis that occurs in the renal epithelium in these diseases.

### Example 20

### FAT10 is Upregulated in Other Forms of Progressive Chronic Kidney Disease

Tubulointerstitial disease is closely associated with prognosis in several renal diseases, including diabetic nephropathy and hyptertensive nephrosclerosis (diseases which cause over 70% of ESRD in the U.S.), and IgA nephropathy (the most common cause of glomerulonephritis worldwide).

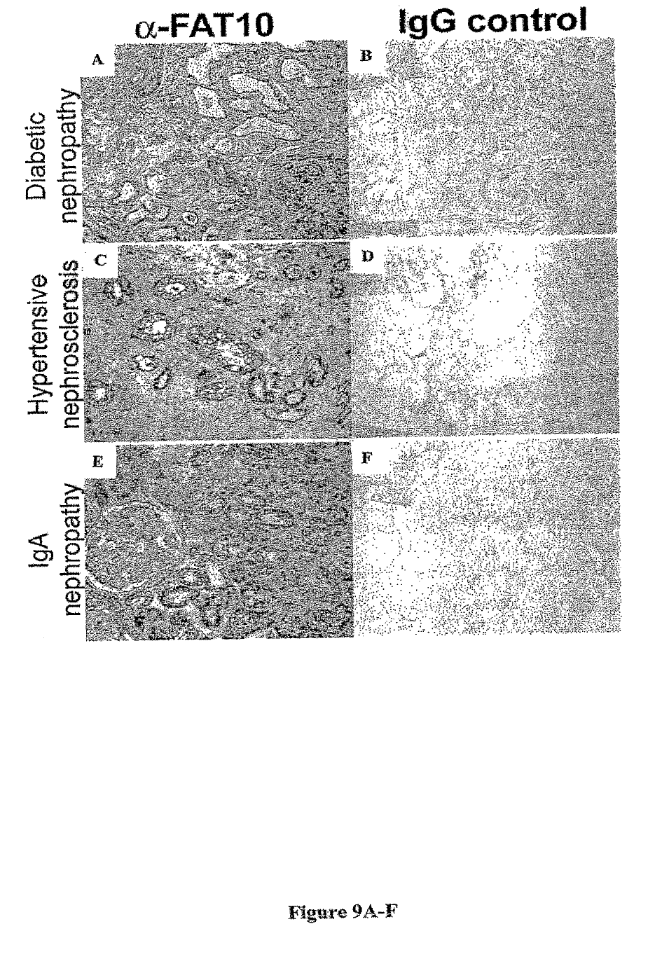

Immunohistochemistry was performed to determine if FAT10 is expressed in renal biopsy specimens from patients with those diseases. A striking expression of FAT10 was detected in 15/15 specimens from patients with each of these diseases (5 patients with each disease). FAT10 was detected predominantly in RTEC, with some glomerular expression, especially in diabetic nephropathy (FIG. 9). Therefore, FAT10 may have a role in the pathogenesis of many renal diseases in addition to HIVAN.

### Example 21

### Nonsynonymous SNPs in FAT10 and Haplotypes

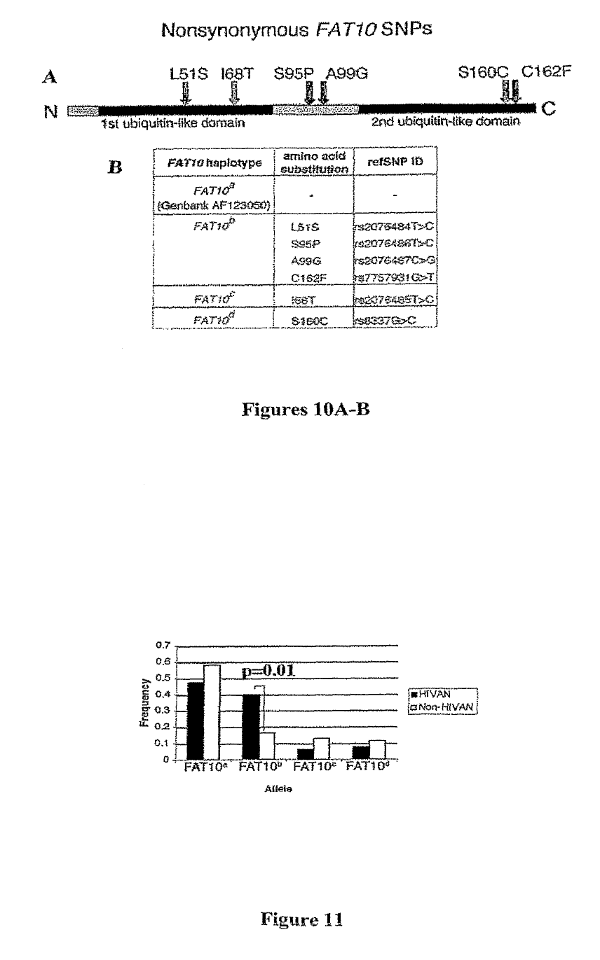

When FAT10 was first cloned from the HPT-1 cell line by reverse transcriptase PCR, it was found that the coding sequence contained four nonsynonymous (encode amino acid substitutions) SNPs as compared to SEQ ID NO: 1. Upon further inquiry, it was found that each of these SNPs had been previously annotated in the NCBI dbSNP database. To determine if these SNPs were inherited independently or whether some inherited as haplotypes, the second exon of FAT10 (containing all nonsynonymous SNPs) was sequenced in all patient samples in the Mount Sinai HIV Renal Biopsy Repository. Analysis of over 100 samples revealed that there are six nonsynomous SNPs that are inherited as four haplotypes of FAT10: FAT10^{a} (SEQ ID NO:1); FAT10^{b}, which is SEQ ID NO:1 with amino acid substitutions L51S, S95P, A99G, and C162F; FAT10^{c}, which is SEQ ID NO:1 with the amino acid substitution I68T; and FAT10^{d}, which is SEQ ID NO:1 with the amino acid substitution S160C (FIGS. 10A-B).

### Example 22

### The FAT10^{b} Allele is Associated with Susceptibility to HIVAN

To determine if any of the four FAT10 alleles are associated with an increased risk of developing HIVAN, the frequency of each allele was compared in patients with HIVAN and HIV-1 seropositive patients with nonHIVAN renal disease using samples from the Mount Sinai HIV Renal Biopsy Repository (40 patients with HIVAN, 35 patients with other forms of renal disease). It was found that FAT10^{b} was significantly more prevalent in patients with HIVAN (FIG. 11) even after controlling for ethnicity and sex using Mantel-Haenszel testing (2-sided p value=0.01, manuscript in preparation). There was only 1 Caucasian patient in this series.

### Example 23

### FAT10 Alleles Differ in their Ability to Induce Apoptosis

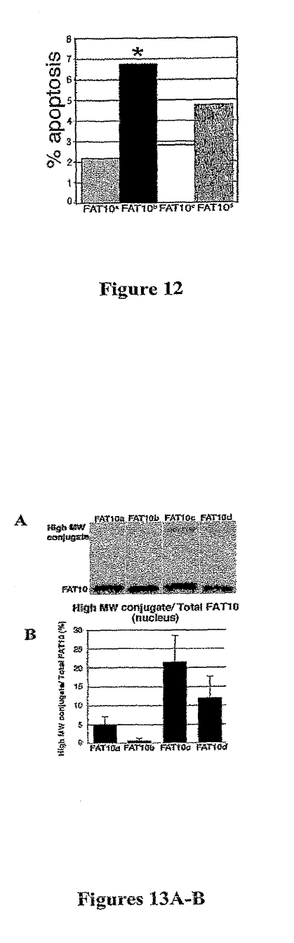

Since it was shown that FAT10 expression induces apoptosis in HPT-1 cells, it was next studied whether the FAT10 alleles differ in their ability to induce apoptosis in this human RTEC line. Each allele was cloned into the pVVPW/BE-FAT10 vector (FIG. 2D). These vectors were then transfected into HPT-1 cells and stained with annexin V and propidium iodide 24 hours after transfection to label apoptotic and necrotic cells. Preliminary analysis of these cells by flow cytometry suggested that the FAT10^{b} allele is the most potent apoptosis-inducing allele in HPT-1 cells (FIG. 12).

### Example 24

### FAT10 Alleles Differ in their Ability to form Covalent Conjugates

Other investigators have shown that the ability of FAT10 to induce apoptosis is dependent upon its ability to become covalently attached to an unknown protein(s) (Raasi et al., “The Ubiquitin-Like Protein FAT10 Forms Covalent Conjugates and Induces Apoptosis,” J. Biol. Chem. 276(38):35334-43 (2001), which is hereby incorporated by reference in its entirety). It was therefore studied whether the FAT10 alleles differ in their ability to form covalent conjugates. 293T cells were transfected with Xpress-tagged FAT10 and nuclear and mitochondrial fractions were isolated after 24 hours, subjected to SDS-PAGE, and western blotting was performed using anti-Xpress antibodies. Nuclear FAT10^{b} formed significantly less high-molecular weight covalent conjugates than the other alleles and FAT10^{C} formed significantly more high molecular weight conjugate than the other alleles (FIGS. 13A-B). The data were similar in mitochondrial fractions. Hipp et al. demonstrated that both FAT10 and its high molecular weight conjugate are rapidly degraded by the proteasome (Hipp et al., “FAT10, A Ubiquitin-Independent Signal for Proteasomal Degradation,” Mol. Cell. Biol. 25(9):3483-91 (2005), which is hereby incorporated by reference in its entirety). However, the use of the proteasome inhibitor MG132 did not significantly affect the nuclear or mitochondrial accumulation of FAT10 or its conjugate in these studies.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of treating or preventing renal disease in a subject, said method comprising:
administering to the subject an agent which inhibits FAT10 expression in the subject under conditions effective to prevent or treat renal disease.

2. The method according to claim 1, wherein the renal disease is characterized by dysregulated apoptosis of renal tubular epithelial cells.

3. The method according to claim 1, wherein the renal disease is selected from the group consisting of autosomal dominant polycystic kidney disease, HIV-associated nephropathy, diabetic nephropathy, hypertensive nephrosclerosis, IgA nephropathy, and acute rejection of transplanted kidneys.

4. The method according to claim 1, wherein the renal disease is treated.

5. The method according to claim 1, wherein the renal disease is prevented.

6. The method according to claim 1, wherein the subject is human.

7. The method according to claim 1, wherein the subject is infected with HIV-1.

8. The method according to claim 1, wherein the agent is an antibody raised against a peptide encoded by the FAT10 gene.

9. The method according to claim 8, wherein the peptide encoded by the FAT10 gene is selected from the group consisting of APNASCLCVHVRSE (SEQ ID NO:3) and DANPYDSVKKIKEVHR (SEQ ID NO:4).

10. The method according to claim 1, wherein said administering is carried out orally, by inhalation, by intranasal instillation, topically, transdermally, parenterally, subcutaneously, intravenously, by intrapleural instillation, intraperitoneally, intraventricularly, intralesionally, by application to mucous membranes, or by implantation of a sustained release vehicle adjacent to affected cells.

11. A method of screening for agents effective in treating or preventing renal disease, said method comprising:
providing one or more candidate compounds;
contacting the one or more candidate compounds with a FAT10 gene under conditions effective to express the FAT10 gene in the absence of the one or more candidate compounds; and
identifying candidate compounds which inhibit FAT10 expression as agents potentially useful in treating or preventing renal disease.

12. The method according to claim 11, wherein said contacting is carried out in vitro.

13. The method according to claim 11, wherein said contacting is carried out in vivo.

14. A method of identifying the ability of a test compound to inhibit expression of FAT10, said method comprising:
providing a cell transfected with a DNA construct comprising:
an isolated nucleic acid molecule encoding a FAT10 protein or polypeptide;
a reporter gene; and
5′ and 3′ regulatory regions that allow transcription and translation of the nucleic acid molecule encoding the FAT10 protein or polypeptide and the reporter gene;
incubating a test compound with the transfected cell;
measuring the level of said reporter gene expressed in said cell; and
comparing the level of expression of the reporter gene expressed in the transgenic cell in the absence of the test compound with the expression of the reporter gene in the presence of the test compound, wherein a decrease in reporter gene expression level indicates the ability of the test compound to inhibit FAT10 expression.

15. The method according to claim 14, wherein the test compound is a peptide, peptidomimetic, or small molecule.


SEQUENCE LISTING (17 sequences)

SEQ ID NO: 1 - protein, 165 aa, Homo sapiens
MAPNASCLCVHVRSEEWDLMTFDANPYDSVKKIKEHVRSKTKVPVQDQVLLLGSKILKPRRSLSSYGIDKEKTIHLTLKVVKPSDEELPLFLVESGDEAKRHLLQVRRSSSVAQVKAMIETKTGIIPETQIVTCNGKRLEDGKMMADYGIRKGNLLFLASYCIGG

SEQ ID NO: 2 - DNA, 971 bp, Homo sapiens
gattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatgtaagctgcatgcaaagtcctttttctgtctctggtttctggccccttgtctgcagagatggctcccaatgcttcctgcctctgtgtgcatgtccgttccgaggaatgggatttaatgacctttgatgccaacccatatgacagcgtgaaaaaaatcaaagaacatgtccggtctaagaccaaggttcctgtgcaggaccaggttcttttgctgggctccaagatcttaaagccacggagaagcctctcatcttacggcattgacaaagagaagaccatccaccttaccctgaaagtggtgaagcccagtgatgaggagctgcccttgtttcttgtggagtcaggtgatgaggcaaagaggcacctcctccaggtgcgaaggtccagctcagtggcacaagtgaaagcaatgatcgagactaagacgggtataatccctgagacccagattgtgacttgcaatggaaagagactggaagatgggaagatgatggcagattacggcatcagaaagggcaacttactcttcctggcatcttattgtattggagggtgaccaccctgggcatggggtgttggcaggggtcaaaaagcttatttcttttaatctcttactcaacgaacacatcttctgatgatttcccaaaattaatgagaatgagatgagtagagtaagatttgggtgggatgggtaggatgaagtatattgcccaactctatgtttctttgattctaacacaattaattaagtgacatgatttttactaatgtattactgagactagtaaataaatttttaagccaa

SEQ ID NO: 3 - protein, 14 aa, Homo sapiens
APNASCLCVHVRSE

SEQ ID NO: 4 - protein, 16 aa, Homo sapiens
DANPYDSVKKIKEHVR

SEQ ID NO: 5 - DNA, 20 bp, Artificial
  Synthetic
gatcttaaagccacggagaa

SEQ ID NO: 6 - DNA, 20 bp, Artificial
  Synthetic
catcccacccaaatcttact

SEQ ID NO: 7 - DNA, 34 bp, Artificial
  Synthetic
cgggatcccaccatggcttctgtccgcacctgtg

SEQ ID NO: 8 - DNA, 34 bp, Artificial
  Synthetic
ggaattcggttatcccccagtgcagtgtgttgtc

SEQ ID NO: 9 - DNA, 33 bp, Artificial
  Synthetic
cgggatccaccatggctcccaatgcttcctgcc

SEQ ID NO: 10 - DNA, 36 bp, Artificial
  Synthetic
ggaattcggttaccctccaatacaataagatgccag

SEQ ID NO: 11 - DNA, 61 bp, Artificial
  Synthetic
gatcctgcttcctgcctctgtgtgttgatatccgcacacagaggcaggaagcattttttgg

SEQ ID NO: 12 - DNA, 61 bp, Artificial
  Sythetic
aattccaaaaaatgcttcctgcctctgtgtgcggatatcaacacacagaggcaggaagcag

SEQ ID NO: 13 - DNA, 61 bp, Artificial
  Synthetic
gatccgaacatgtccggtctaagattgatatccgtcttagaccggacatgttcttttttgg

SEQ ID NO: 14 - DNA, 61 bp, Artificial
  Synthetic
aattccaaaaaagaacatgtccggtctaagacggatatcaatcttagaccggacatgttcg

SEQ ID NO: 15 - DNA, 60 bp, Artificial
  Synthetic
gatccgtgcgttgctagtaccaacttcaagagagttggtactagcaacgcacttttttgg

SEQ ID NO: 16 - DNA, 60 bp, Artificial
  Synthetic
aattccaaaaaagtgcgttgctagtaccaactctcttgaagttggtactagcaacgcacg

SEQ ID NO: 17 - DNA, 61 bp, Artificial; identical to SEQ ID NO: 11
  Synthetic